(19) United States
(12) Patent Application Publication
(10) Pub. No.: US 2007/0269384 A1
(43) Pub. Date: Nov. 22, 2007

(54) Use of perfluoroalkyl-containing metal complexes as contrast media for diagnosis of Alzheimer's disease

(76) Inventors: Bernd Misselwitz, Glienicke (DE); Hanns-Joachim Weinmann, Berlin (DE); Joerg Meding, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/797,898

(22) Filed: May 8, 2007

Related U.S. Application Data
(60) Provisional application No. 60/798,709, filed on May 9, 2006.

(30) Foreign Application Priority Data
May 9, 2006 (DE) 10 2006 021 495.1

(51) Int. Cl. A61K 49/10

(52) U.S. Cl. 424/9.363; 424/9.364

(57) ABSTRACT

The invention relates to the use of metal complexes that contain at least one perfluorinated alkyl radical and at least one chelating agent radical and at least one metal ion equivalent of the atomic numbers 21-29, 31-33, 37-39, 42-44, 49 or 57-83, as well as salts thereof for the production of a diagnostic agent for visualizing amyloid-containing plaques.

This application claims the benefit of the filing date of U.S. Provisional Application Ser. No. 60/798,709 filed May 9, 2006, which is incorporated by reference herein.

The invention relates to the subject that is characterized in the claims, i.e., the use of metal complexes that contain at least one perfluorinated alkyl radical and at least one chelating agent radical and at least one metal ion equivalent of the atomic numbers 21-29, 31-33, 37-39, 42-44, 49 or 57-83, as well as salts thereof for the production of a diagnostic agent for the diagnosis of Alzheimer's disease.

Alzheimer's disease is an organocerebral disease that is characterized by the slowly progressing degeneration of nerve cells and nerve cell contacts. In the USA, about 4 million humans suffer from Alzheimer's disease. Annual costs of about 100 billion dollars result therefrom in the USA alone. Worldwide, about 20 million humans suffer from dementia diseases, of which about 15 million suffer from Alzheimer's disease. Based on the increasing live expectancy, it is thus calculated that the number of those affected will grow to 45 million by the year 2050.

Alzheimer's disease is thus the most common form of senile dementia. It begins with slight memory disorders and results in the further course of the disease in the loss of large portions of memory, in reduction of vocabulary to a few words, to space and time disorientation, to motor disorders, as well as often to aconuresis and anal incontinence. The last stage in most cases is characterized by total need of care. The epidemiology of Alzheimer's disease shows that age is the greatest risk factor and that in the case of correspondingly increasing age, almost any human can contract Alzheimer's disease. In most cases, the disease is apparent only after the 65^{th} year, but the onset occurs as early as between the 40^{th} and 60^{th} year. Alzheimer's disease is therefore an ever greatly increasing social and financial problem in particular for Western industrial countries with high life expectancy.

The clinical symptoms of Alzheimer's disease are produced by an increasing loss of nerve cells. The result of this is the shrinking of the brain by up to 20%, which can be made visible in the middle and advanced disease stages by imaging processes such as computer tomography (CT) or magnetic resonance tomography (NRT). In addition, the visualization of the regional cerebral blood supply or the regional cerebral metabolism can contribute to the safety of the diagnosis. There is no reliable laboratory test for Alzheimer's disease to date.

With definitive certainty, the diagnosis of Alzheimer's disease can be made by the study of the brain only after death (post-mortem). In this case, the identification of the characteristic pathological albumen deposits (amyloid plaques), which are to be found between the nerve cells, is used as the most reliable parameter. The latter consist of a central amyloid nucleus, which is surrounded by pathologically altered nerve cell appendages and supporting cells. In numerous patents, the amyloid is also deposited in the wall of small blood vessels. These amyloid plaques are essentially the single previously known connection between pathological findings and disease symptoms. The main component of these plaques are aggregates of the β-amyloid-peptide (Aβ-peptide or βA4-peptide), which comprises 39 to 43, but usually 40 or 42 amino acid radicals. Whether the plaques are a result of the disease or conversely the disease symptoms are based on the presence of the plaques is a controversial topic. The latter model, the so-called “amyloid hypothesis,” became more complete and more consistent in recent years, although definitive evidence or counter-evidence could not be produced until now (D. J. Selkoe, Science 1997, 275, 630-631).

Diagnostic processes, which are based on the detection of amyloid plaques (quick in-vitro or in-vivo tests for estimating the amount, elapsed time and quality of the amyloid structures) still do not exist at this time. Also, a reliable differentiation of Alzheimer's disease from other dementia diseases cannot be made with the momentarily available diagnostic in vivo processes. The existing approaches for in vivo imaging are unsatisfactory. Alternative methods, e.g., near-infrared imaging (NIR), positron emission tomography (PET) and optical imaging are still in a very early stage and are limited in applicability. An overview on existing in vivo imaging approaches for diagnosis of Alzheimer's is found in, for example, Mathis, C. A. et al., 2004, Curr. Pharmac. Design, 10: 1469-1492 or Gremlich, H.-U. et al., 2005, Siemens Medical Solutions, September 2005: 64-70 and Hintersteiner, M. et al., 2005, Nature Biotech., 23: 577-583.

The magnetic resonance tomography (MRT) is common as the diagnostic cross-sectional process and makes possible a very good spatial resolution. Therefore, an MR-based process for differential diagnosis of Alzheimer's disease would be advantageous and of great medical use. MRT studies performed to date (without contrast media or with use of extracellular contrast media) would not result, however, in any satisfactory results, since the contrast achieved in the cerebral lesions was not sufficient and the contrast media used were not able to overcome the intact blood-brain barrier.

The differential diagnosis of Alzheimer's disease is increasingly important, however, with respect to the available treatment methods. In this case, the early diagnosis of the disease is of special importance to ensure early start-up of treatment. In addition, the monitoring of the success of treatment is also of great importance for adequate therapy.

There is therefore a clear medicinal need for a very readily compatible, as little invasive a process as possible for early in vivo diagnosis of Alzheimer's disease in humans.

The object of this invention was therefore to make available contrast media for the diagnosis of Alzheimer's disease that meet the necessary criteria: very good systemic compatibility, overcoming the blood-brain barrier for build-up in the amyloid plaques, sufficiently long diagnostic windows for reliable diagnosis of the disease.

It was now found that, surprisingly enough, certain perfluoroalkyl-containing metal complexes, as contrast media, in particular in MR imaging, are very well suited for diagnosis of Alzheimer's disease.

The invention relates to the use of metal complexes that contain

at least one perfluorinated alkyl radical, and

at least one chelating agent radical, and

at least one metal ion equivalent of the atomic numbers 21-29, 31-33, 37-39, 42-44, 49 or 57-83, as well as salts thereof

for the production of a diagnostic agent for the visualization of amyloid-containing plaques.

The use of such metal complexes or salts thereof, which contain 1 perfluorinated alkyl radical and 1 or 2 chelating agent radicals, is preferred.

In a preferred embodiment, the metal complexes that can be used according to the invention contain a perfluorinated alkyl radical and a chelating agent radical.

In another preferred embodiment, the metal complexes that can be used according to the invention contain a perfluorinated alky radical and 2 chelating agent radicals.

A “perfluorinated alkyl radical” in terms of this invention is an alkyl radical with 4-30 C atoms, which is perfluorinated, i.e., all hydrogen atoms of the radical are substituted by fluorine atoms. In a preferred embodiment, the metal complexes contain a perfluorinated alkyl radical with 6-20 C atoms.

In an especially preferred embodiment, the perfluorinated alkyl radical is linear.

Linear perfluorinated alkyl radicals with 6-14 C atoms are especially preferred; linear perfluorinated alkyl radicals with 7, 8, 9, 10, 11 or 12 C atoms are quite especially preferred.

In another preferred embodiment, the perfluorinated alky radical is branched. Branched perfluorinated alkyl radicals with 8-16 C atoms are especially preferred; branched perfluorinated alkyl radicals with 9, 10, 11, 12, 13 or 14 C atoms are quite especially preferred.

In an especially preferred embodiment, the stability constant of the metal complexes is at least 10¹⁵, preferably at least 10¹⁸. The stability constant is determined as described in Martell, A. E.; Motekaitis, R. J. (The Determination and Use of Stability Constants, 2^{nd} Ed.; VCH: New York, 1992).

Compounds with these properties have already been described as diagnostic agents in MRI imaging in the unpublished DE102005033902.6-44, the unpublished DE 102005033903.4, the WO 02/14309, the WO 02/013875, the WO 00/56723, the WO 99/01161, WO 97/26017 and WO 02/13874.

The substances that are usable according to the invention were previously proposed for various applications, such as, e.g., for visualizing the bloodstream (angiography) or the lymph stream (lymphography), for diagnosis of arteriosclerosis, intravascular thrombosis or tumors, as well as for infarction imaging.

There are no indications in the prior art, however, that the substances can also be used for the diagnosis of Alzheimer's disease.

The use of metal complexes or salts thereof as MRI contrast media is quite especially preferred.

If the contrast medium is to be used in the ¹H-MRI process, the metal ion must be paramagnetic in the metal complexes that can be used according to the invention.

The metal complexes can contain a signaling group. “Signaling group” in terms of this invention is a group that allows the metal complex to be used as a contrast medium or tracer in other imaging processes in addition to ¹H-MRI imaging processes and/or the x-ray diagnosis.

If the contrast medium is to be used, for example, in the ¹⁹F-MR imaging process, the metal complex or salt thereof must contain at least one ¹⁹F atom as a signaling group; preferably the perfluoroalkyl radical contains at least one ¹⁹F atom; in particular all fluorine atoms of the perfluoroalkyl radical are ¹⁹F.

Other suitable imaging processes in which the metal complexes according to the invention can be used are, for example, SPECT, PET, radiodiagnosis and optical imaging.

Such metal complexes that contain a signaling group can be used, for example, in optical imaging. The optical signal can be any signal that can be detected, including the transmission or absorption of light of a specific wavelength (e.g., near infrared, fluorescence absorption or phosphorescence absorption or phosphorescence emission, reflection, changes of absorption amplitude or absorption maxima). In general, the optical signal is an NIR (near infrared) or fluorescence emission spectrum. Methods for detection in vivo comprise, for example, catheters, which are equipped with a suitable optical detector. In Example 2, a metal complex that contains a signaling group that fluoresces is disclosed.

In particular, the contrast media that can be used according to the invention are suitable for distinguishing Alzheimer's disease from other dementia diseases.

In particular, the contrast media that can be used according to the invention are suitable for visualizing amyloid plaques.

Perfluoroalkyl-containing metal complexes that are especially suitable for use according to the invention are amphiphilic compounds that have a perfluoroalkyl side chain in the molecule as a nonpolar part, which perfluoroalkyl side chain optionally is connected to the total molecule via a lipophilic linker. The polar portion of the compounds according to the invention is formed by one or more metal complexes and optionally present additional polar groups.

In aqueous systems, these amphiphilic molecules show the properties that are characteristic of standard surfactants (such as, e.g., sodium dodecyl sulfate, SDS). Thus, they reduce the surface tension of the water. By tensiometry, the so-called CMC (critical micelle formation concentration in mol/l) can be determined. To this end, the surface tension is determined based on the concentration of the substance to be measured. The CMC can be calculated from the course of the surface tension (c) function that is obtained. The critical micelle formation concentration of the compounds according to the invention should be <10⁻³ mol/l, preferably <10⁻⁴ mol/l.

The amphiphilic compounds according to the invention are combined in solution and are present as aggregates. The size (2 Rh) of such aggregates (e.g., micelles, rods, oblates, etc.) can be determined with the aid of photon-correlation spectroscopy (PCS).

As a second criterion for preferably usable metal complexes, therefore, the hydrodynamic micelle diameter 2 Rh, which must be >1 nm, is used. Those perfluoroalkyl-containing metal complexes, whose 2 Rh≧3 nm, quite especially preferably >4 nm, are especially suitable according to the invention.

Both the determination of CMC and the photon correlation spectroscopy are described in H.-D. Dorfler, “Grenzflächen- and Kolloidchemie [Interface and Colloid Chemistry],” Weinheim, New York, Basel, Cambridge, Tokyo, VSH 1994.

As a third criterion for especially suitable MR contrast media, the proton relaxivity (relaxivity) in plasma (R₁) at 40° C. and a field strength of 0.47 Tesla is used. The relaxivity, which is indicated in [l/mmol*s], is the quantitative measurement for the shortening of the relaxation time T₁ of the protons. For the purpose according to the invention, the relaxivity must be as high as possible and must be >10 l/mmol*s, preferably >13 l/mmol*s, especially preferably >15 l/mmol*s.

The relaxivity R₁ [l/mmol*s] of the MR contrast media according to the invention was determined with the Minispec P 20 device of the Bruker Company. The measurements were made at 40° C. and a field strength of 0.47 Tesla. 8 measuring points were recorded in each T1 sequence (180°-T1-90°, inversion recovery). As a medium, bovine plasma of the Kraeber Company was used. The contrast medium concentrations [mmol/l] in the preparations were between 0.30 and 1.16.

In particular, therefore, those metal complexes for the ¹H-MRI imaging processes are suitable that are characterized in that the metal complexes or salts thereof have

a critical micelle formation concentration<10⁻³ mol/l,

a hydrodynamic micelle diameter (2 Rh)>1 nm, and

a proton relaxivity in the plasma (R₁)>10 l/mmol*s.

Metal complexes are preferably used that overcome the blood-brain barrier. Such metal complexes can be administered, for example, i.v. The intravenous form of administration is preferred for this application. It is also possible, however, to administer the substances that can be used according to the invention orally or systemically. In addition, the metal complexes can also be administered locally or administered intrathecally.

In an embodiment of this invention, the compounds of general formula I
R^{F}-L-K I
are used as preferred compounds,
in which
R^{F} is a perfluorinated, straight-chain or branched carbon chain with the formula —CₙF₂ₙE, in which
   E represents a terminal fluorine, chlorine, bromine, iodine or hydrogen atom, and n stands for the numbers 4-30,
L means a direct bond, a methylene group, an —NHCO group, a group 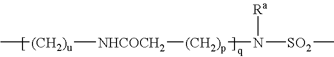
   whereby p means the numbers 0 to 10, q and u, independently of one another, mean the number 0 or 1, and
      R^{a} means a hydrogen atom, a methyl group, a benzyl group, a phenyl group, a —CH₂—OH group, a CH₂OCH₃ group, a —CH₂—CO₂H group, or a C₂—C₁₅ chain, which optionally is interrupted by 1 to 3 oxygen atoms, 1 to 2>CO groups or an optionally substituted aryl group and/or is substituted by 1 to 4 hydroxyl groups, 1 to 2 C₁-C₄-alkoxy groups, 1 to 2 carboxy groups, or a group —SO₃H—,
   or a straight-chain, branched, saturated or unsaturated C₂-C₃₀-carbon chain, which optionally contains 1 to 10 oxygen atoms, 1 to 3 —NR^{a} groups, 1 to 2 sulfur atoms, a piperazine, a —CONR^{a} group, one to six —NR^{a}CO groups, an —SO₂ group, an —NR^{a}—CO₂ group, 1 to 2 —CO groups, 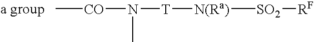
   or 1 to 2 optionally substituted aryls, and/or is interrupted by these groups, and/or is optionally substituted by 1 to 3 —OR^{a} groups, 1 to 2 oxo groups, 1 to 2 —NH—COR^{a} groups, 1 to 2-CONHR^{a} groups, 1 to 2 —(CH₂)ₚ—CO₂H groups, or 1 to 2 —(CH₂)ₚ—(O)_{q}—CH₂CH₂—R^{F} groups, whereby
   R^{a}, R^{F} and p and q have the above-indicated meanings, and
   T means a C₂-C₁₀-chain, which optionally is interrupted by 1 to 2 oxygen atoms or 1 to 2 —NHCO groups,
K stands for a complexing agent or for a metal complex or salts thereof of organic and/or inorganic bases or amino acids or amino acid amides, specifically for a complexing agent or complex of general formula II 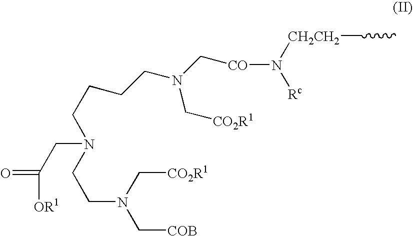
   in which R^{c}, R¹ and B are independent of one another, and
   R^{c} has the meaning of R^{a} or means —(CH₂)ₘ-L-R^{F}, whereby
      m is 0, 1 or 2, and L and R^{F} have the above-mentioned meaning,
   R¹, independently of one another, means a hydrogen atom or a metal ion equivalent of the atomic numbers 21-29, 31-33, 37-39, 42-44, 49 or 57-83,
   B means —OR¹ or 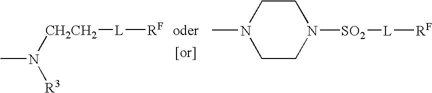
      whereby R¹, L, R^{F} and R^{c} have the above-mentioned meanings, or
   for a complexing agent or complex of general formula III 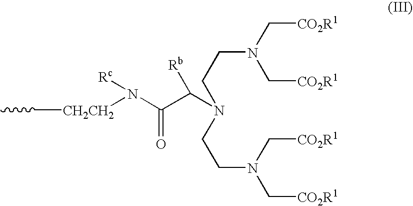
   in which R^{c} and R¹ have the above-mentioned meanings,
   R^{b} has the meaning of R^{a}
or
   for a complexing agent or complex of general formula IV 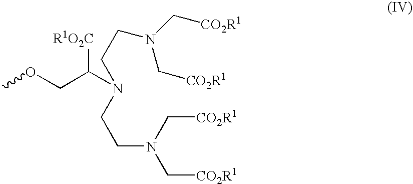
   in which R¹ has the above-mentioned meaning,
or
   for a complexing agent or complex of general formula V 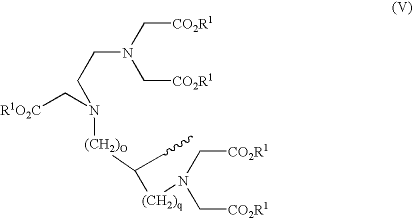
   in which R¹ has the above-mentioned meaning, and o and q stand for the numbers 0 or 1 and produces the sum o+q=1,
or
   for a complexing agent or complex of general formula VI 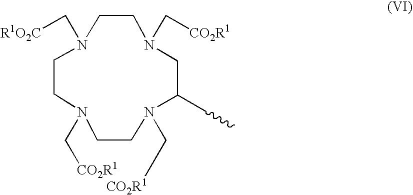
   in which R¹ has the above-mentioned meaning
or
   for a complexing agent or complex of general formula VII 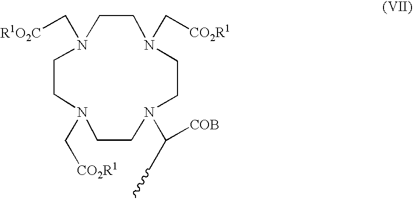
   in which R¹ and B have the above-mentioned meanings,
or
   for a complexing agent or complex of general formula VIII 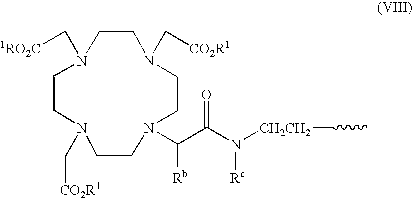
   in which R^{c} and R¹ have the above-mentioned meanings, and R^{b} has the above-mentioned meaning of R^{a},
or
   for a complexing agent or complex of general formula IX 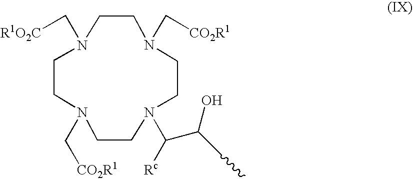
   in which R^{c} and R¹ have the above-mentioned meanings,
or
   for a complexing agent or complex of general formula X 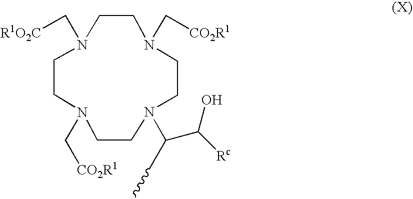
   in which R^{c} and R¹ have the above-mentioned meanings,
or
   for a complexing agent or complex of general formula XI 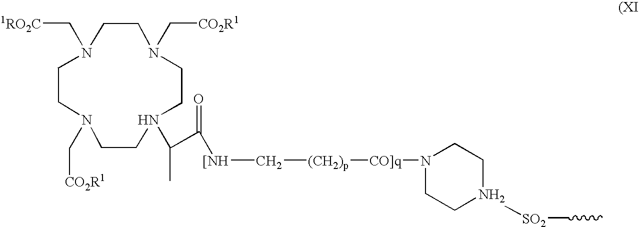
   in which R¹, p and q have the above-mentioned meaning, and R^{b} has the meaning of R^{a},
or
   for a complexing agent or complex of general formula XII 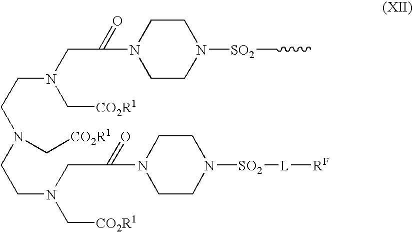
   in which L, R^{F} and Z¹ have the above-mentioned meanings,
or
   for a complexing agent or complex of general formula XIII 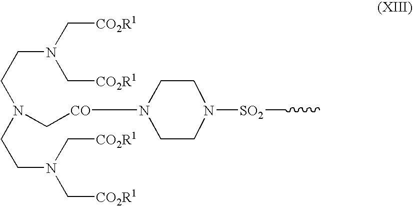
in which R¹ has the above-mentioned meaning.

In a preferred embodiment, compounds of general formula I are used in which L stands for
α-CH₂-β
α-CH₂CH₂-β
α-(CH₂)ₛ-βs=3-15
α-CH₂—O—CH₂CH₂-β
α-CH₂—(O—CH₂—CH₂-)ₜ-β with t=2-6
α-CH₂—NH—CO—β
α-CH₂—NH—CO—CH₂—N(CH₂COOH)—SO₂-β
α-CH₂—NH—CO—CH₂—N(C₂H₅)—SO₂-β
α-CH₂—NH—CO—CH₂—N(C₁₀H₂₁)—SO₂-β
α-CH₂—NH—CO—CH₂—N(C₆H₁₃)—SO₂-β
α-CH₂—NH—CO—(CH₂)₁₀—N(C₂H₅)—SO₂-β
α-CH₂—NH—CO—CH₂—N(—CH₂—C₆H₅)—SO₂-β
α-CH₂—NH—CO—CH₂—N(—CH₂—CH₂—OH)SO₂-β
α-CH₂—NHCO—(CH₂)₁₀—S—CH₂CH₂-β
α-CH₂NHCOCH₂—O—CH₂CH₂-β
α-CH₂NHCO(CH₂)₁₀—O—CH₂CH₂-β
α-CH₂—C₆H₄—O—CH₂CH₂-β
α-CH₂—O—CH₂—C(CH₂—OCH₂CH₂—C₆F₁₃)₂—CH₂—OCH₂—CH₂-β

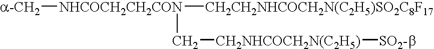

α-CH₂—O—CH₂—CH(OC₁₀OH₂₁)—CH₂—O—CH₂CH₂-β
α-(CH₂NHCO)₄—CH₂O—CH₂CH₂-β
α-(CH₂NHCO)₃—CH₂O—CH₂CH₂-β
α-CH₂—OCH₂C(CH₂OH)₂—CH₂—O—CH₂CH₂-β

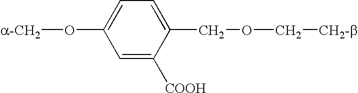

α-CH₂NHCOCH₂N(C₆H₅)—SO₂-β
α-NHCO—CH₂—CH₂-β
α-NHCO-CH₂—O—CH₂CH₂-β
α-NH—CO-β
α-NH—CO—CH₂—N(CH₂COOH)—SO₂-β
α-NH—CO—CH₂—N(C₂H₅)—SO₂-β
α-NH—CO—CH₂—N(C₁₀H₂₁)—SO₂-β
α-NH—CO—CH₂—N(C₆H₁₃)—SO₂-β
α-NH—CO—(CH₂)₁₀—N(C₂H₅)—SO₂-β
α-NH—CO—CH₂—N(—CH₂—C₆H₅)—SO₂-β
α-NH—CO—CH₂—N(—CH₂—CH₂—OH)SO₂-β
α-NH—CO—CH₂-β
α-CH₂—O—C₆H₄—O—CH₂—CH₂-β
α-CH₂—C₆H₄—O—CH₂—CH₂-β
α-N(C₂H₅)—SO₂-β
α-N(C₆H₅)—SO₂-β
α-N(C₁₀H₂₁)—SO₂-β
α-N(C₆H₁₃)—SO₂-β
α-N(C₂H₄OH)—SO₂-β
α-N(CH₂COOH)—SO₂-β
α-N(CH₂C₆H₅)—SO₂-β
α-N-[CH(CH₂OH)₂]-SO₂-β
α-N-[CH(CH₂OH)CH(CH₂OH)]—SO₂-β
and in which α represents the binding site to the complexing agent or metal complex K and β represents the binding site to the fluorine radical.

In another preferred embodiment, compounds of formula I are used in which n in the formula —CₙF₂ₙE stands for the numbers 4-15 and/or E in this formula means a fluorine atom.

In an especially preferred embodiment, the following compounds can be used:

Gadolinium complex of 10-[1-methyl-2-oxo-3-aza-5-oxo-{4-perfluoro-octylsulfonyl-piperazin-1-yl}-pentyl]-1,4,7-tris(carboxymethyl)-1,4,7,10-tetraazacyclododecane,

Gadolinium complex of 10-[2-hydroxy-4-aza-5-oxo-7-oxa-10,10,11,11,12,12, 13,13,14,14,15,15,16,16,17,17-heptadecafluoroheptacecyl]-1,4,7-tris(carboxymethyl)-1,4,7,10-tetraazacyclododecane,

Gadolinium complex of 10-[2-hydroxy-4-aza-5,9-dioxo-9-{4-perfluorooctyl)-piperazin-1-yl}-nonyl]-1,4,7-tris(carboxymethyl)-1,4,7,10-tetraazacyclododecane,

Gadolinium complex of 10-[2-hydroxy-4-aza-5-oxo-7-aza-7-(perfluorooctyl-sulfonyl)-nonyl]-1,4,7-tris(carboxymethyl)-1,4,7,10-tetraazacyclododecane,

Gadolinium complex of 10-[2-hydroxy-4-oxa-1H,1H,2H,3H,3H,5H,5H,6H,6H-perfluoro-tetradecyl]-1,4,7-tris(carboxymethyl)-1,4,7,10-tetraazacyclododecane,

Gadolinium complex of 10-[2-hydroxy-4-aza-5-oxo-7-oxa-10,10,11,11, 12,12,13,13,14,14,15,15,16,16,17,17,18,18,19,19-henicosafluoro-nonadecyl]-1,4,7-tris(carboxymethyl)-1,4,7,10-tetraazacyclododecane,

Gadolinium complex of 10-[2-hydroxy-4-aza-5-oxo-11-aza-1-(perfluoro-octylsulfonyl)-tridecyl]-1-4-7-tris(carboxymethyl) 1,4,7,10-tetraazacyclododecane,

Gadolinium complex of 10-[2-hydroxy-4-aza-5-oxo-7-aza-7-(perfluoro-octylsulfonyl)-8-phenyl-octyl]-1-4-7-tris(carboxymethyl)-1,4,7,10-tetraazacyclododecane

These are known compounds that are described in WO 97/26017. Also, their production can be seen from this WO publication. It has been shown, surprisingly enough, that these compounds are also very well suited as contrast media for diagnosis of Alzheimer's disease. As quite especially preferred compounds, the metal complexes MK 2, 3 and 4 as well as MK 8, 9, 10 and 11 (cf. also Table 1) are used, which are especially suitable for use in MR imaging.

In another embodiment of this invention, as preferred compounds of those perfluoroalkyl-containing metal complexes, the compounds of general formula Ia
A-R^{F} (Ia)
are used,
in which
A is a molecule portion that contains 2-6 metal complexes that are bonded directly or via a linker to a nitrogen atom of an annular skeleton chain, and
R^{F} is a perfluorinated, straight-chain or branched carbon chain with the formula —CₙF₂ₙE, in which
   E is a terminal fluorine, chlorine, bromine, iodine, or hydrogen atom, and n stands for the numbers 4-30,
whereby the molecule portion A has the following structure: 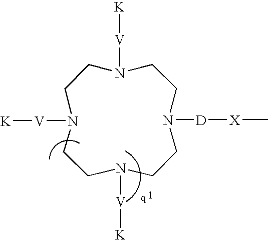
whereby
q¹ is a number 0, 1, 2 or 3,
K stands for a complexing agent or metal complex or their salts of organic and/or inorganic bases or amino acids or amino acid amides,
X is a direct bond to the perfluoroalkyl group, a phenylene group or a C₁-C₁₀-alkylene chain, which optionally contains 1-15 oxygen atoms, 1-5 sulfur atoms, 1-10 carbonyl groups, 1-10 (NR^{d}) groups, 1-2 NR^{d}SO₂ groups, 1-10 CONR^{d} groups, 1 piperidine group, 1-3 SO₂ groups, or 1-2 phenylene groups or optionally is substituted by 1-3 R^{F} radicals, in which R^{d} stands for a hydrogen atom, a phenyl group, a benzyl group or a C₁-C₁₅-alkyl group, which optionally contains 1-2 NHCO groups, 1-2 CO groups, or 1-5 oxygen atoms, and optionally is substituted by 1-5 hydroxy radicals, 1-5 methoxy radicals, 1-3 carboxy radicals, or 1-3 R^{F} radicals,
V is a direct bond or a chain of general formula Ia or IIa: 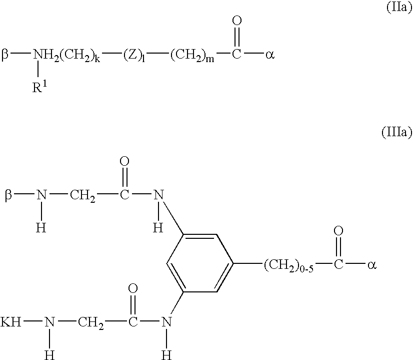
in which
R^{e} is a hydrogen atom, a phenyl group, a benzyl group, or a C₁-C₇ alkyl group, which optionally is substituted with a carboxy group, a methoxy group or a hydroxyl group,
W is a direct bond, a polyglycol ether group with up to 5 glycol units or a molecule portion of general formula IVa
   —CH(R^{h})— (IVa)
   in which R^{h} is a C₁-C₇-carboxylic acid, a phenyl group, a benzyl group or a —(CH₂)₁₋₅—NH—K group,
α represents the bond to the nitrogen atom of the skeleton chain, β represents the bond to the complexing agent or the metal complex K,
and in which the variables k and m stand for natural numbers between 0 and 10 and l stands for 0 or 1,
   and whereby
D is a CO group or SO₂ group.

In this case, compounds of general formula Ia, in which q is the number 1, are preferably used.

In another preferred embodiment, compounds of general formula Ia are used, in which the molecule portion X is an alkylene chain, which contains 1-10 CH₂CH₂O groups or 1-5 COCH₂NH groups, a direct bond or one of the following structures 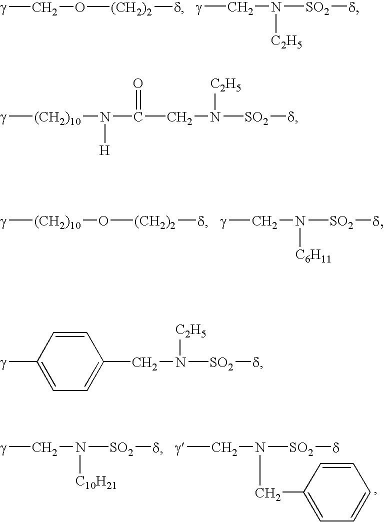
whereby

γ binds to D and δ binds to R^{F}.

In another preferred embodiment, compounds of general formula Ia are used, in which V is a molecule portion with one of the following structures

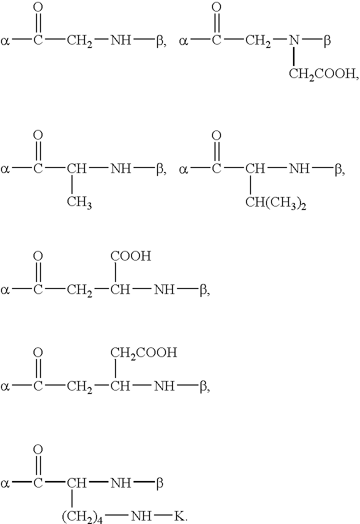

In addition, compounds of general formula Ia are preferred, in which K represents a complex of general formula Va, VIa, VIIa or VIIIa 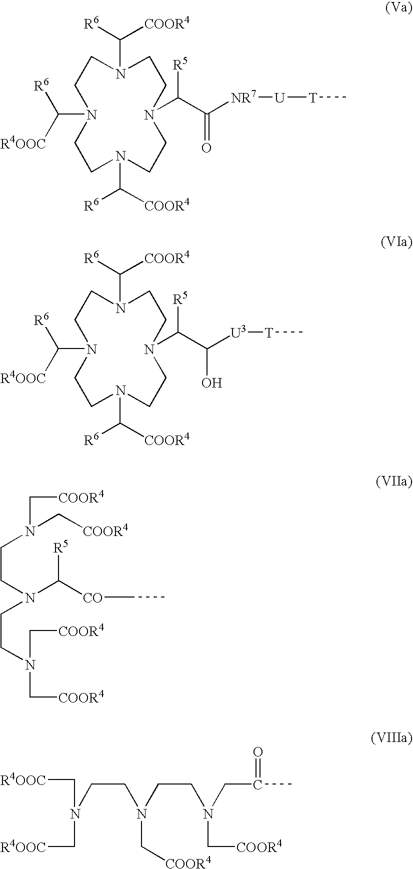
whereby
R⁴, independently of one another, is a hydrogen atom or a metal ion equivalent of the elements of atomic numbers 23-29, 42-46 or 58-70,
R⁵ is a hydrogen atom or a straight-chain, branched, saturated or unsaturated C₁-C₃₀-alkyl chain, which optionally is substituted by 1-5 hydroxy groups, 1-3 carboxy groups or 1 phenyl group and/or optionally is interrupted by 1-10 oxygen atoms, 1 phenylene group or 1 phenylenoxy group,
R⁶ is a hydrogen atom, a straight-chain or branched C₁-C₇-alkyl radical, a phenyl radical or benzyl radical,
R⁷ is a hydrogen atom, a methyl group or ethyl group, which optionally is substituted by a hydroxy group or a carboxy group,
U³ is a straight-chain, branched, saturated or unsaturated C₁-C₂₀-alkylene group that optionally contains 1-5 imino groups, 1-3 phenylene groups, 1-3 phenylenoxy groups, 1-3 phenylenimino groups, 1-5 amide groups, 1-2 hydrazide groups, 1-5 carbonyl groups, 1-5 ethylenoxy groups, 1 urea group, 1 thiourea group, 1-2 carboxyalkylimino groups, 1-2 ester groups, 1-10 oxygen atoms, 1-5 sulfur atoms and/or 1-5 nitrogen atoms, and/or optionally is substituted by 1-5 hydroxy groups, 1-2 mercapto groups, 1-5 oxo groups, 1-5 thioxo groups, 1-3 carboxy groups, 1-5 carboxyalkyl groups, 1-5 ester groups and/or 1-3 amino groups, whereby the optionally contained phenylene groups can be substituted by 1-2 carboxy groups, 1-2 sulfone groups or 1-2 hydroxy groups,
T¹ stands for a —CO-β, —NHCO-β or —NHCS-β group, whereby β represents the binding site to V.

In this case, the C₁-C₂₀-alkylene chain that stands for U³ preferably contains the groups —CH₂NHCO—, —NHCOCH₂O—, —NHCOCH₂OC₆H₄—, —N(CH₂CO₂H)—, —CH₂OCH₂—, —NHCOCH₂C₆H₄—, —NHCSNHC₆H₄—, —CH₂OC₆H₄—, —CH₂CH₂O-and/or it is substituted by the groups —COOH, —CH₂COOH.

U³ quite especially preferably stands for a —CH₂—, —CH₂CH₂—, —CH₂CH₂CH₂—, —C₆H₄—, —C₆H₁₀—, —CH₂C₆H₄—, —CH₂NHCOCH₂CH(CH₂CO₂H)—C₆H₄—, —CH₂NHCOCH₂OCH₂—, or —CH₂NHCOCH₂C₆H₄ group.

The compounds of general formula Ia, in which K has one of the following structures, are quite especially preferred:

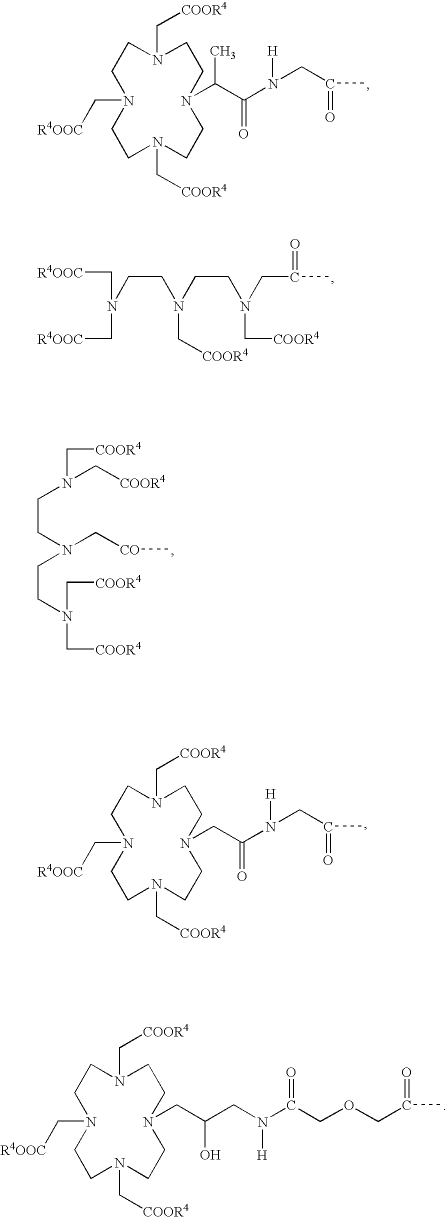

In another preferred embodiment, the perfluoroalkyl chain R^{F} is —C₆F₁₃, —C₈F₁₇, —C₁₀F₂₁ or —C₁₂F₂₅.

Quite especially preferred is the use of the gadolinium complex of 1,4,7-tris{1,4,7-tris(N-(carboxylatomethyl)-10-[N-1-methyl-3,6-diaza-2,5,8-trioxooctane-1,8-diyl)]-1,4,7,10-tetraazacyclododecane, Gd complex}-10-[N-2H, 2H, 4H, 4H, 5H, 5H-3-oxa-perfluoro-tridecanoyl]-1,4,7,10-tetraazacyclododecane.

These compounds are known and described in WO 99/01161. Their use as contrast media, which are suitable in particular for application in MR imaging, was previously not described for diagnosis of Alzheimer's. Of these compounds, quite especially preferably the metal complex MK 12 (cf. Table 1) is used.

In another preferred embodiment of the invention, the macrocyclic perfluoroalkyl compounds of general formula Ib 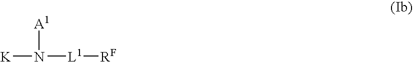
in which

K means a complexing agent or a metal complex of general formula IIb,

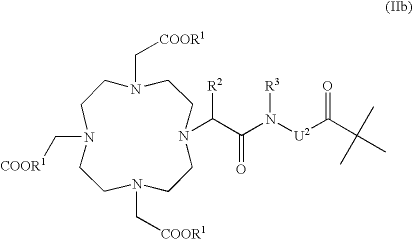

whereby
R¹ stands for a hydrogen atom or a metal ion equivalent of the atomic numbers 21-29, 31-33, 37-39, 42-44, 49 or 57-83,
R² and R³ stand for a hydrogen atom, a C₁-C₇-alkyl group, a benzyl group, a phenyl group, —CH₂OH or —CH₂—OCH₃, and
U² stands for the radical L¹, whereby L¹ and U², independently of one another, can be the same or different,
A¹ means a hydrogen atom, a straight-chain or branched C₁-C₃₀-alkyl group, which optionally is interrupted by 1-15 oxygen atoms, and/or optionally is substituted by 1-10 hydroxy groups, 1-2 COOH groups, a phenyl group, a benzyl group and/or 1-5 OR^{g} groups, with R^{g} in the meaning of a hydrogen atom or a C₁-C₇-alkyl radical, or -L¹-R^{F},
L¹ means a straight-chain or branched C₁-C₃₀-alkylene group, which optionally is interrupted by 1-10 oxygen atoms, 1-5 —NH—CO groups, 1-5 —CO—NH groups, by a phenylene group that optionally is substituted by a COOH group, 1-3 sulfur atoms, 1-2-N(B¹)-SO₂ groups, and/or 1-2 —SO₂—N(B¹) groups with B¹ in the meaning of A¹, an NHCO group, a CONH group, an N(B¹)—SO₂— group, or an —SO₂—N(B¹) group and/or optionally is substituted with the radical R^{F}, and
R^{F} means a straight-chain or branched perfluorinated alkyl radical of the formula CₙF₂ₙE,
   whereby n stands for the numbers 4-30, and
      E stands for a terminal fluorine atom, chlorine atom, bromine atom, iodine atom, or a hydrogen atom,
and optionally existing acid groups that are optionally present as salts of organic and/or inorganic bases or amino acids or amino acid amides can be used, as they and their production are disclosed and defined in WO 00/56723.

In a preferred embodiment, compounds of general formula Ib are used, in which R², R³ and R^{g}, independently of one another, mean hydrogen or a C₁-C₄-alkyl group.

In addition, the compounds of general formula Ib can be used, in which A¹ means hydrogen, a C₁-C₁₅-alkyl radical,

the radicals C₂H₄—O—CH₃, C₃H₆—O—CH₃,
C₂H₄—O—(C₂H₄—O)ₜ—C₂H₄—OH,
C₂H₄—O—(C₂H₄—O)ₜ—C₂H₄—OCH₃,
C₂H₄OH, C₃H₆OH, C₄H₈OH, C₅H₁₀OH, C₆H₁₂OH, C₇H₁₄OH,
CH(OH)CH₂OH,
CH(OH)CH(OH)CH₂OH, CH₂[CH(OH)]ᵤ¹CH₂OH,
CH[CH₂(OH)]CH(OH)CH₂OH,
C₂H₄CH(OH)CH₂OH,
(CH₂)ₛCOOH,
C₂H₄—O—(C₂H₄—O)ₜ—CH₂COOH or
C₂H₄—O—(C₂H₄—O)ₜ—C₂H₄—CₙF₂ₙE,

whereby
s stands for the whole numbers 1 to 15,
t stands for the whole numbers 0 to 13,
u¹ stands for the whole numbers 1 to 10,
n stands for the whole numbers 4 to 20, and
E stands for a hydrogen atom, fluorine atom, chlorine atom, bromine atom or iodine atom, as well as, if possible, the branched isomers thereof.

In addition, the compounds of general formula Ib are preferably used, in which
A¹ means hydrogen, C₁-C₁₀-alkyl,
   C₂H₄—O—CH₃, C₃H₆—O—CH₃,
   C₂H₄—O—(C₂H₄—O)ₜ—C₂H₄—OH, C₂H₄—O—(C₂H₄—O)ₜ—C₂H₄—OCH₃,
   C₂H₄OH, C₃H₆OH,
   CH₂[CH(OH)]_{y}CH₂OH,
   CH[CH₂(OH)]CH(OH)CH₂OH,
   (CH₂)_{w}COOH,
   C₂H₄—O—(C₂H₄—O)ₜ—CH₂COOH,
   C₂H₄—O—(C₂H₄—O)ₜ—C₂H₄—CₙF₂ₙE,

whereby
x stands for the whole numbers 0 to 5,
y stands for the whole numbers 1 to 6,
w stands for the whole numbers 1 to 10,
n stands for the whole numbers 4 to 15, and
E stands for a fluorine atom, as well as, if possible, the branched isomers thereof.

In addition, the compounds of general formula Ib are preferably used, in which L¹ means
α-(CH₂)ₛ-β
α-CH₂—CH₂—(O—CH₂—CH₂—)_{y}-β
α-CH₂—(O—CH₂—CH₂—)_{y}-β
α-CH₂—NH—CO-β
α-CH₂—CH₂—NH—SO₂-β
α-CH₂—NH—CO—CH₂—N(CH₂COOH)—SO₂-β
α-CH₂—NH—CO—CH₂—N(C₂H₅)—SO₂-β
α-CH₂—NH—CO—CH₂—N(C₁₀H₂₁)—SO₂-β
α-CH₂—NH—CO—CH₂—N(C₆H₁₃)—SO₂-β
α-CH₂—NH—CO—(CH₂)₁₀—N(C₂H₅)—SO₂-β
α-CH₂—NH—CO—CH₂—N(—CH₂—C₆H₅)—SO₂-β
α-CH₂—NH—CO—CH₂—N(—CH₂—CH₂—OH)SO₂-β
α-CH₂—NHCO—(CH₂)₁₀—S—CH₂CH₂-β
α-CH₂NHCOCH₂—O—CH₂CH₂-β
α-CH₂—CH₂NHCOCH₂—O—CH₂CH₂-β
α-CH₂—(CH₂—CH₂—O)ₜ—(CH₂)₃NHCO—CH₂—O—CH₂CH₂-β
α-CH₂NHCO(CH₂)₁₀—CH₂CH₂-β
α-CH₂CH₂NHCO(CH₂)₁₀—O—CH₂CH₂-β
α-CH₂-C₆H₄—O—CH₂CH₂-β whereby the phenylene group is 1,4- or 1,3-linked
α-CH₂—O—CH₂—C(CH₂—OCH₂CH₂—C₆F₁₃)₂—CH₂—OCH₂—CH₂-β
α-CH₂—NHCOCH₂CH₂CON—CH₂CH₂NHCOCH₂N(C₂H₅)SO₂C₈F₁₇β
α-CH₂—CH₂NHCOCH₂N(C₂H₅)—SO₂-β
α-CH₂—O—CH₂—CH(OC₁₀H₂₁)—CH₂—O—CH₂CH₂-β
α-(CH₂NHCO)₄—CH₂O—CH₂CH₂-β
α-(CH₂NHCO)₃—CH₂O—CH₂CH₂-β
α-CH₂—OCH₂C(CH₂OH)₂—CH₂—O—CH₂CH₂-β

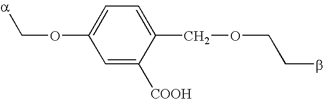

α-CH₂NHCOCH₂N(C₆H₅)—SO₂-β
α-NHCO—CH₂—CH₂-β
α-NHCO-CH₂—O—CH₂CH₂-β
α-NH—CO-β
α-NH—CO—CH₂—N(CH₂COOH)—SO₂-β
α-NH—CO—CH₂‘N(C₂H₅)—SO₂-β
α-NH—CO—CH₂—N(C₁₀H₂₁)—SO₂-β
α-NH—CO—CH₂—N(C₆H₁₃)—SO₂-β
α-NH—CO—(CH₂)₁₀—N(C₂H₅)—SO₂-β
α-NH—CO—CH₂—N(—CH₂—C₆H₅)—SO₂-β
α-NH—CO—CH₂—N(—CH₂—CH₂—OH)SO₂-β
α-NH—CO—CH₂-β
α-CH₂—O—C₆H₄—O—CH₂—CH₂-β
α-CH₂—C₆H₄—O—CH₂—CH₂-β
α-N(C₂H₅)—SO₂-β
α-N(C₆H₅)—SO₂-β
α-N(C₁₀H₂₁)—SO₂-β
α-N(C₆H₁₃)—SO₂-β
α-N(C₂H₄OH)—SO₂-β
α-N(CH₂COOH)-SO₂-β
α-N(CH₂C₆H₅)—SO₂-β
α-N-[CH(CH₂OH)₂]—SO₂-β
α-N-[CH(CH₂OH)CH(OH)(CH₂OH)]-SO₂-β,

whereby

s stands for the whole numbers 1 to 15, and

y stands for the whole numbers 1 to 16.

In addition, the compounds of general formula Ib are preferably used, in which L¹ means

α-CH₂—O—CH₂CH₂-β

α-CH₂—CH₂—(O—CH₂—CH₂—)_{y}-β

α-CH₂—(O—CH₂—CH₂-)_{y}-β

α-CH₂—CH₂—NH—SO₂-β, Example 10

α-CH₂NHCOCH₂—O—CH₂CH₂-β,

α-CH₂—CH₂NHCOCH₂—O—CH₂CH₂-β,

α-CH₂—(CH₂—CH₂—O)_{y}—(CH₂)₃NHCO—CH₂—O—CH₂CH₂-β,

α-CH₂NHCO(CH₂)₁₀—O—CH₂CH₂-β,

α-CH₂CH₂NHCO(CH₂)₁₀—O—CH₂CH₂-β,

α-CH₂—O—CH₂—CH(OC₁₀H₂₁)—CH₂—O—CH₂CH₂-β,

α-CH₂—O—C₆H₄—O—CH₂—CH₂-β or

α-CH₂—C₆H₄—O—CH₂—CH₂-β,

whereby

y stands for the whole numbers 1 to 6.

In addition, the compounds of general formula Ib are preferably used, in which R^{F} means a straight-chain or branched perfluorinated alkyl radical of formula CₙF₂ₙE, whereby n stands for the numbers 4 to 15, and E stands for a terminal fluorine atom.

The following compounds are quite especially preferably used:
1,4,7-Tris(carboxylatomethyl)-10-(3-aza-4-oxo-hexan-5-ylic)-acid-N-(2,3-dihydroxypropyl)-N-(1H, 1H, 2H, 2H, 4H, 4H, 5H, 5H-3-oxa)-perfluoro-tridecyl)-amide]-1,4,7,10-tetraazacyclododecane, gadolinium complex
1,4,7-Tris(carboxylatomethyl)-10-{(3-aza-4-oxo-hexan-5-ylic)-acid-N-(3,6,9,12,15-pentaoxa)-hexadecyl)-(1H, 1H, 2H, 2H, 4H, 4H, 5H, 5H-3-oxa)-perfluorotridecyl]-amide}-1,4,7,10-tetraazacyclododecane, gadolinium complex
1,4,7-Tris(carboxylatomethyl)-10-{(3-aza-4-oxo-hexan-5-ylic)-acid-N-5-hydroxy-3-oxa-pentyl)-N-(1H, 1H, 2H, 2H, 4H, 4H, 5H, 5H-3-oxa)-perfluorotridecyl]-amide}-1,4,7,10-tetraazacyclododecane, gadolinium complex
1,4,7-Tris(carboxylatomethyl)-10-{(3-aza-4-oxo-hexan-5-ylic)-acid-[N-3,6,9,1 5-tetraoxa-12-aza-15-oxo-C₁₇-C₂₆-hepta-decafluoro)hexacosyl]-amide}-1,4,7,10-tetraazacyclododecane, gadolinium complex
1,4,7-Tris(carboxylatomethyl)-10-[(3-aza-4-oxo-hexan-5-ylic]-acid-N-(2-methoxyethyl)-N-(1H, 1H, 2H, 2H, 4H, 4H, 5H, 5H-3-oxa)-perfluorotridecyl]-amide}-1,4,7,10-tetraazacyclododecane, gadolinium complex.

According to the invention, the metal complexes MK 17, MK 18, MK 19, MK 21 and MK 23 (cf. Table 1), which are suitable in particular for use in MR imaging, are quite especially preferably used.

These compounds of general formula Ib are very well suited as contrast media, in particular MRI contrast media, for diagnosis of Alzheimer's.

In another preferred embodiment of the invention, the perfluoroalkyl-containing complexes can be used with sugar radicals of general formula Ic (see also WO 02/13874) 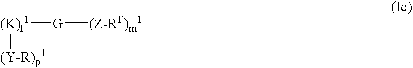
in which
R represents a monosaccharide radical or oligosaccharide radical that is bonded via the 1-OH position or 1-SH position,
R^{F} is a perfluorinated, straight-chain or branched carbon chain with the formula —CₙF₂ₙE, in which E represents a terminal fluorine atom, chlorine atom, bromine atom, iodine atom or hydrogen atom, and n stands for the numbers 4-30,
K stands for a metal complex of general formula IIc,

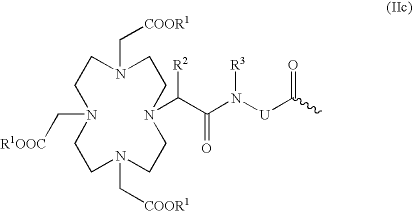

in which
R¹ means a hydrogen atom or a metal ion equivalent of the atomic numbers 21-29, 31-33, 37-39, 42-44, 49 or 57-83, provided that at least two R¹ stand for metal ion equivalents,
R² and R³, independently of one another, represent hydrogen, C₁-C₇-alkyl, benzyl, phenyl,
   —CH₂OH or —CH₂OCH₃, and
U represents —C₆H₄—O—CH₂-ω-, —(CH₂)₁₋₅-ω, a phenylene group, —CH₂—NHCO—CH₂—CH(CH₂COOH)—C₆H₄-ω-, —C₆H₄—(OCH₂CH₂)₀₋₁—N(CH₂COOH)—CH₂-ω or a C₁-C₁₂-alkylene group or C₇-C₁₂-C₆H₄—O group that optionally is interrupted by one or more oxygen atoms, 1 to 3 —NHCO groups or 1 to 3-CONH groups and/or is substituted with 1 to 3 —(CH₂)₀₋₅COOH groups, whereby o stands for the binding site to —CO—, or
of general formula IIIc 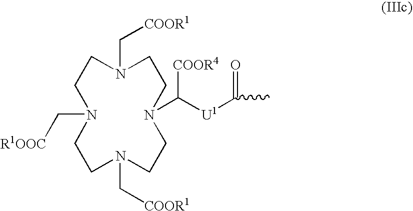
in which R¹ has the above-mentioned meaning, R⁴ represents hydrogen or a metal ion equivalent that is mentioned under R¹, and U¹ represents —C₆H₄—O—CH₂-ω-, whereby ω means the binding site to —CO—,
   or of general formula IVc

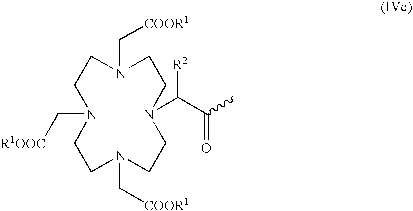

in which R¹ and R² have the above-mentioned meaning,
or of general formula VcA or VcB

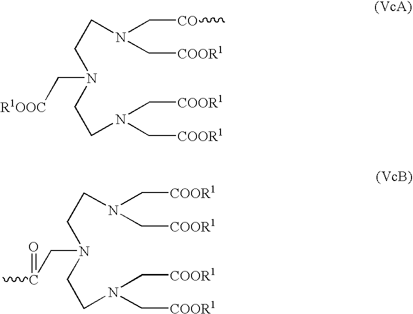

in which R¹ has the above-mentioned meaning,
or of general formula VIc 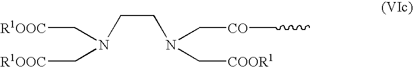
in which R¹ has the above-mentioned meaning,
or of general formula VIIc

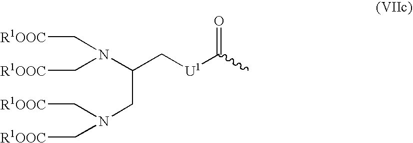

in which R¹ has the above-mentioned meaning, and

U¹ represents —C₆H₄—O—CH₂-ω-, whereby ω means the binding site to —CO—
or of general formula VIIIc 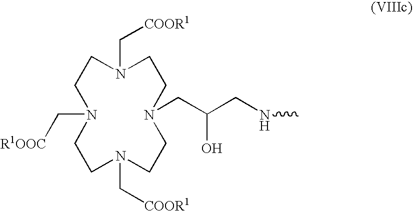
in which R¹ has the above-mentioned meaning,
and in radical K, optionally present free acid groups optionally can be present as salts of organic and/or inorganic bases or amino acids or amino acid amides,
G stands for the case that K means the metal complexes IIc to VIIc, a radical that is functionalized in at least three places selected from the radicals a) to j) below 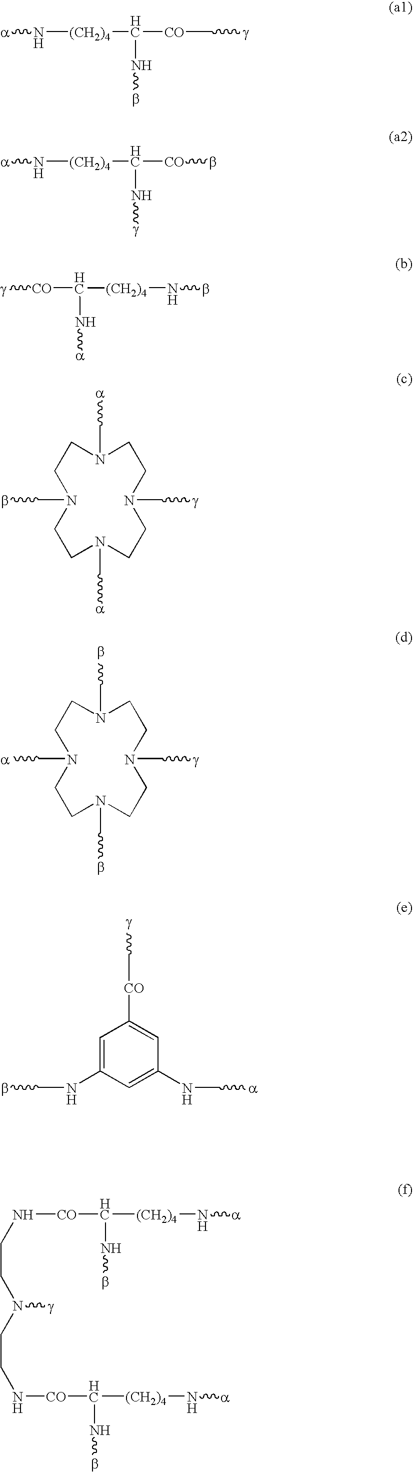 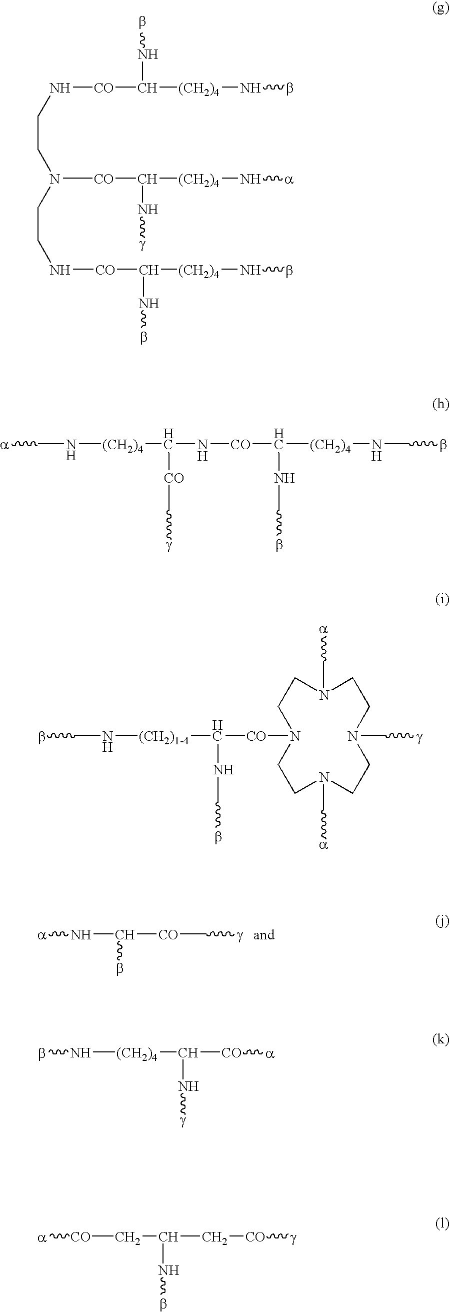
G stands for the case that K means the metal complex VIIIc, a radical that is functionalized in at least three places and that is selected from k) or l), 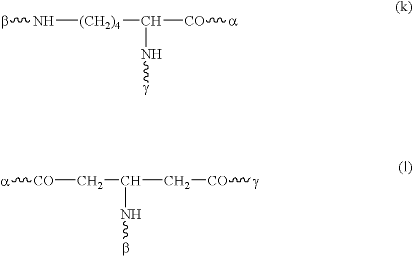
whereby α means the binding site of G to the complex K, β is the binding site of G to the radical Y, and γ is the binding site of G to the radical Z,
Y means —CH₂—, δ-(CH₂)₁₋₅CO-β, β-(CH₂)₁₋₅CO-δ, δ-CH₂—CHOH—CO-β or δ-CH(CHOH—CH₂OH)—CHOH—CHOH—CO-β, whereby δ represents the binding site to the sugar radical R, and δ is the binding site to the radical G.
Z stands for 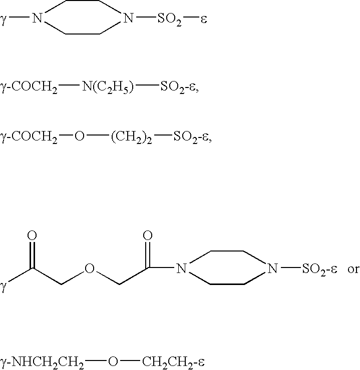
   whereby γ represents the binding site of Z to radical G, and ε means the binding site of Z to the perfluorinated radical R^{F}, and
l¹, m¹, independently of one another, mean the whole numbers 1 or 2, and
p¹ means the whole numbers 1 to 4.

In this case, these are known compounds that are described in WO 02/143097. Also, their production can be found in this WO publication. Surprisingly enough, it has been shown that these compounds also are very well suited as contrast media, in particular MRI contrast media, for diagnosis of Alzheimer's disease.

In a preferred embodiment, compounds of general formula Ic are used in which R represents a monosaccharide radical with 5 to 6 C atoms or its deoxy compound, preferably glucose, mannose, or galactose.

In a preferred embodiment, compounds of general formula Ic are used, in which R² and R³, independently of one another, mean hydrogen or C₁-C₄-alkyl, and/or E in the formula —CₙF₂ₙE means a fluorine atom.

In a preferred embodiment, compounds of general formula Ic are used, in which G represents the lysine radical (a) or (b).

In a preferred embodiment, compounds of general formula Ic are used, in which Z means 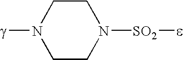
whereby γ represents the binding site of Z to the radical G, and ε means the binding site of Z to the perfluorinated radical R^{F}, and/or Y means δ-CH₂CO-β, whereby δ represents the binding site to the sugar radical R, and β represents the binding site to the radical G.

In a preferred embodiment, compounds of general formula Ic are used, in which U in the metal complex K represents —CH₂— or —C₆H₄—O—CH₂-ω, whereby ω stands for the binding site to —CO—.

As a quite especially preferred compound of general formula Ic, the gadolinium complex of 6-N-[1,4,7-tris(carboxylatomethyl)-1,4,7,10-tetraazacyclododecane-10-N-(pentanoyl-3-aza-4-oxo-5-methyl-5-yl)]-2-N-[1-O-α-D-carbonylmethyl-manno-pyranose]-L-lysine-[1-(4-perfluorooctylsulfonyl)-piperazine]-amide is used.

As a quite especially preferred compound of general formula Ic, the metal complex MK 13 of Table 1 is used according to the invention.

In another preferred embodiment of the invention, the perfluoroalkyl-containing complexes with polar radicals of general formula Id (see also WO 02/13874) are used

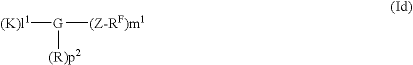

in which
R^{F} is a perfluorinated, straight-chain or branched carbon chain with the formula —CₙF₂ₙE, in which E represents a terminal fluorine atom, chlorine atom, bromine atom, iodine atom or hydrogen atom, and n stands for the numbers 4-30,
K stands for a metal complex of general formula IId,

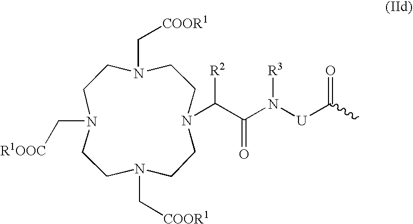

in which
R¹ means a hydrogen atom or a metal ion equivalent of the atomic numbers 23-29, 42-46 or 58-70,
   provided that at least two R¹ stand for metal ion equivalents,
R² and R³, independently of one another, represent hydrogen, C₁-C₇-alkyl, benzyl, phenyl, -CH₂OH or -CH₂OCH₃, and
U represents —C₆H₄—O—CH₂-ω-, —(CH₂)₁₋₅-ω, a phenylene group, —CH₂—NHCO—CH₂—CH(CH₂COOH)—C₆H₄-ω-, —C₆H₄—(OCH₂CH₂)₀₋₁-N(CH₂COOH)—CH₂-ω or a C₁-C₁₂-alkylene group or C₇-C₁₂-C₆H₄—O group that optionally is interrupted by one or more oxygen atoms, 1 to 3 NHCO groups or 1 to 3 CONH groups and/or is substituted with 1 to 3 —(CH₂)₀₋₅COOH groups, whereby ω stands for the binding site to —CO—,
or
of general formula IIId 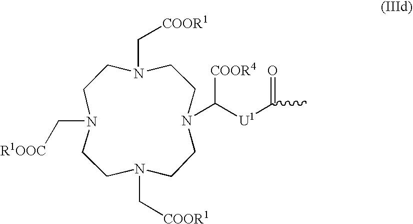
in which R¹ has the above-mentioned meaning, R⁴ represents hydrogen or a metal ion equivalent that is mentioned under R¹ and U¹ represents —C₆H₄—O—CH₂-ω-, whereby ω means the binding site to —CO—
   or of general formula IVd 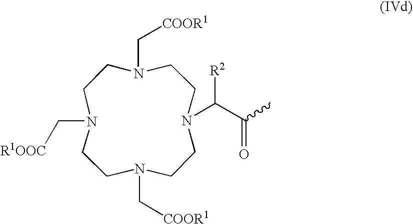
in which R¹ and R² have the above-mentioned meaning
   or of general formula VdA or VdB 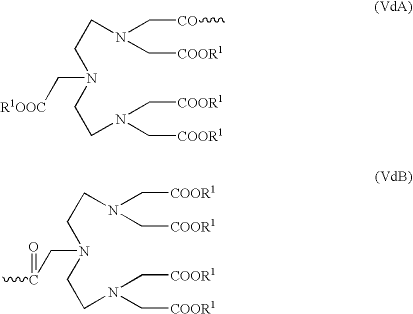
in which R¹ has the above-mentioned meaning,
   or of general formula VId 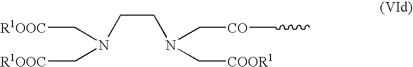
in which R¹ has the above-mentioned meaning,
   or of general formula VIId 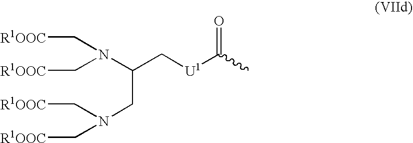
in which R¹ has the above-mentioned meaning, and
U¹ represents —C₆H₄—O—CH₂-ω-, whereby ω means the binding site to —CO— and in radical K, optionally present free acid groups optionally can be present as salts of organic and/or inorganic bases or amino acids or amino acid amides,
G represents a radical, functionalized in at least three places, that is selected from the radicals a) to i) below 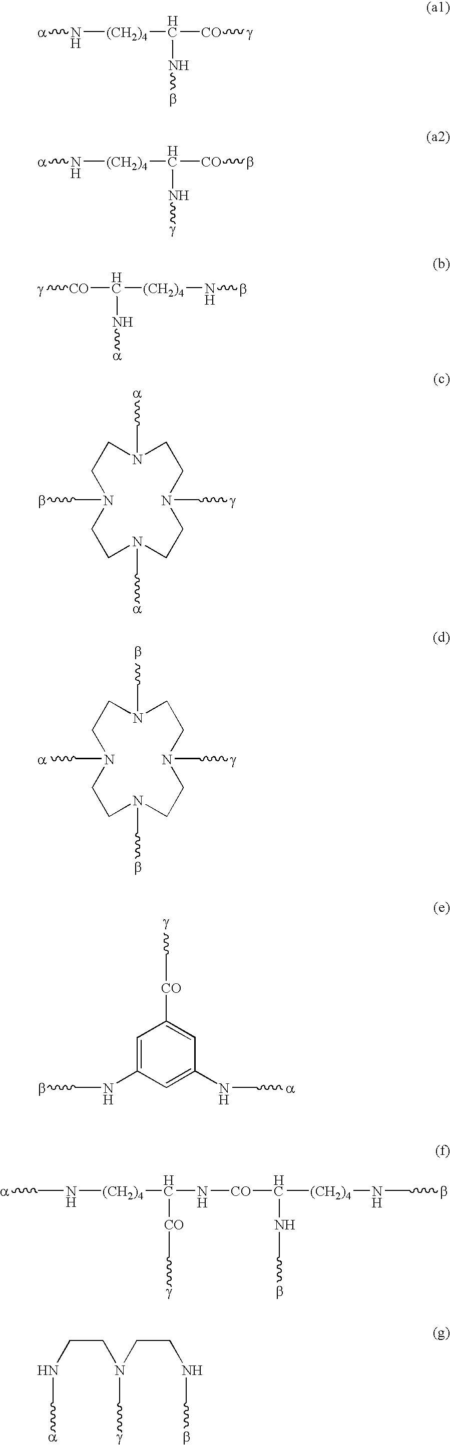 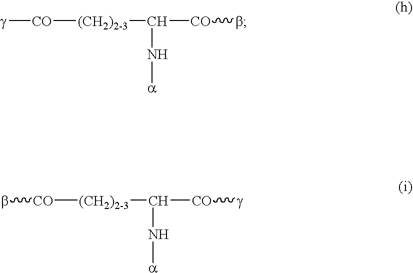
whereby a means the binding site of G to the complex K, β is the binding site of G to the radical R, and γ represents the binding site of G to the radical Z
Z stands for 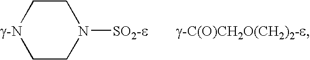
whereby γ represents the binding site of Z to the radical G, and ε means the binding site of Z to the perfluorinated radical R^{F},
R represents a polar radical that is selected from the complexes K of general formulas IId to VIId, whereby R¹ here means a hydrogen atom or a metal ion equivalent of the atomic numbers 20, 23-29, 42-46 or 58-70, and the radicals R², R³, R⁴, U and U¹ have the above-indicated meaning, or
   means the folic acid radical
   or
   means a carbon chain with 2-30 C atoms that is bonded via —CO—, SO₂ or a direct bond to the radical G, straight or branched, saturated or unsaturated,
   optionally interrupted by 1-10 oxygen atoms, 1-5 —NHCO groups, 1-5 —CONH groups, 1-2 sulfur atoms, 1-5 —NH groups or 1-2 phenylene groups, which optionally can be substituted with 1-2 OH groups, 1-2 NH₂ groups, 1-2 COOH groups or 1-2 —SO₃H groups, or optionally substituted with 1-8 OH groups, 1-5 —COOH groups, 1-2 SO₃H groups, 1-5 NH₂ groups, 1-5 C₁-C₄-alkoxy groups, and
l¹, m¹, p², independently of one another, mean the whole number 1 or 2.

In a preferred embodiment, compounds of general formula Id are used, in which K stands for a metal complex of general formula IId, IIId, VdB or VId.

In another preferred embodiment, compounds of general formula Id are used, in which the polar radical R has the meaning of complex K, preferably the complexes K of general formulas IId, IIId, VdA or VIId.

In a preferred embodiment, compounds of general formula Id are used, in which the polar radical R has the following meanings:
—C(O)CH₂CH₂SO₃H
—C(O)CH₂OCH₂CH₂OCH₂CH₂OH
—C(O)CH₂OCH₂CH₂OH
—C(O)CH₂OCH₂CH(OH)CH₂OH
—C(O)CH₂NH—C(O)CH₂COOH
—C(O)CH₂CH(OH)CH₂OH
—C(O)CH₂OCH₂COOH
—SO₂CH₂CH₂COOH
—C(O)—C₆H₃-(m-COOH)₂
—C(O)CH₂O(CH₂)₂—C₆H₃-(m-COOH)₂
—C(O)CH₂O—C₆H₄-m-SO₃H
—C(O)CH₂NHC(O)CH₂NHC(O)CH₂OCH₂COOH
—C(O)CH₂OCH₂CH₂OCH₂COOH
—C(O)CH₂OCH₂CH(OH)CH₂O—CH₂CH₂OH
—C(O)CH₂OCH₂CH(OH)CH₂OCH₂—CH(OH)—CH₂OH
—C(O)CH₂SO₃H
—C(O)CH₂CH₂COOH
—C(O)CH(OH)CH(OH)CH₂OH
—C(O)CH₂O[(CH₂)₂O]₁₋₉—CH₃
—C(O)CH₂O[(CH₂)₂O]₁₋₉—H
—C(O)CH₂OCH(CH₂OH)₂
—C(O)CH₂OCH(CH₂OCH₂COOH)₂
—C(O)—C₆H₃-(m-OCH₂COOH)₂ or
—CO—CH₂O—(CH₂)₂O(CH₂)₂O—(CH₂)₂O(CH₂)₂OCH₃

Especially preferable is —C(O)CH₂O[(CH₂)₂O]₄—CH₃.

In another preferred embodiment, compounds of general formula Id are used, in which the polar radical R is the folic acid radical.

In another preferred embodiment, compounds of general formula Id are used, in which G represents the lysine radical (a) or (b).

In another preferred embodiment, compounds of general formula Id are used, in which U in the metal complex K represents the group —CH₂— or —C₆H₄—O—CH₂-ω, whereby ω stands for the binding site to —CO—.

In an especially preferred embodiment, the gadolinium complex of 2,6-N,N′-bis[1,4,7-tris(carboxylatomethyl)-1,4,7,10-tetraazacyclododecane-10-(pentanoyl-3-aza-4-oxo-5-methyl-5-yl)]-lysine-[1-(4-perfluorooctylsulfonyl-piperazine]-amide is used.

In this case, these are known compounds that are described in WO 02/013875. Also, their production can be found in this WO publication. It has been shown, surprisingly enough, that these compounds also are very well suited as contrast media, in particular MRI contrast media, for diagnosis of Alzheimer's disease.

Especially preferred compounds of general formula Id are those with the macrocyclic compound K of general formula IId, IIId, VdB or VIId.

The metal complex MK 12 of Table 1 is used according to the invention as a quite especially preferred compound of general formula Id.

In another preferred embodiment of the invention, the metal complexes of general formula Ie 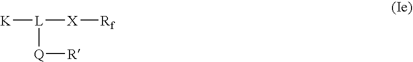
can be used,
in which
R′ either represents a monosaccharide or oligosaccharide radical bonded via the 1-OH, in this case Q has the meaning of a group that is selected from:
   δ-CO—(CH₂)_{n″}-ε
   δ-NH—(CH₂)_{n″}-ε
   δ-(CH₂)_{m″}-ε

whereby

n″ is a whole number from 1 to 5, and

m″ is a whole number from 1 to 6, and

whereby δ indicates the binding site to the linker L, and E the binding site to the radical R′;

or
R′ has one of the following meanings, then Q has the meaning of a direct bond: R means a polar radical that is selected from
   the complexes K of general formulas IIc to Vc, whereby R¹ here means a hydrogen atom or a metal ion equivalent of the atomic numbers 21-29, 31-33, 37-39, 42-44, 49 or 57-83, and the radicals R², R³, R⁴, U and U¹ have the meanings that are indicated below,
      or
   a carbon chain with 1-30 C atoms that is bonded via —CO—, —NR⁷— or a direct bond to the linker L, which can be straight or branched, saturated or unsaturated, and which optionally is interrupted by 1-10 oxygen atoms, 1-5 —NHCO— groups, 1-5 —CONH groups, 1-2 sulfur atoms, 1-5 —NH groups, or 1-2 phenylene groups, which optionally can be substituted with 1-2 OH groups, 1-2 NH₂ groups, 1-2 —COOH groups, or 1-2 —SO₃H groups, and which optionally is substituted with 1-10 OH groups, 1-5 —COOH groups, 1-2 SO₃H groups, 1-5 NH₂ groups, or 1-5 C₁-C₄-alkoxy groups,
      whereby R⁷ means H or C₁-C₄ alkyl,
R_{f} means a perfluorinated, straight-chain or branched carbon chain with the formula —CₙF₂ₙE, in which E represents a terminal fluorine atom, chlorine atom, bromine atom, iodine atom or hydrogen atom, and n stands for the numbers 4-30,
K stands for a metal complex of general formula IIc,

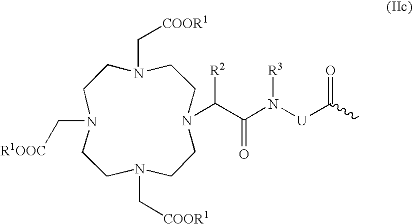

in which
R¹ means a hydrogen atom or a metal ion equivalent of atomic numbers 21-29, 31-33, 37-39, 42-44, 49 or 57-83, provided that at least two R¹ stand for metal ion equivalents,
R² and R³, independently of one another, represent hydrogen, C₁-C₇-alkyl, benzyl, phenyl, —CH₂OH or —CH₂OCH₃, and
U represents —C₆H₄—O—CH₂-ω-, —(CH₂)₁₋₅-ω, a phenylene group, —CH₂—NHCO—CH₂—CH(CH₂COOH)—C₆H₄-ω-, —C₆H₄—(OCH₂CH₂)₀₋₁-N(CH₂COOH)—CH₂-ω or a C₁-C₁₂-alkylene group or —(CH₂)₇₋₁₂—C₆H₄—O group that optionally is interrupted by one or more oxygen atoms, 1 to 3 —NHCO groups or 1 to 3 —CONH groups and/or is substituted by 1 to 3 —(CH₂)₀₋₅COOH groups, whereby ω stands for the binding site to —CO—,
   or of general formula IIIc 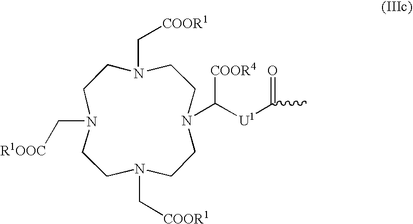
in which R¹ has the above-mentioned meaning, R⁴ represents hydrogen or a metal ion equivalent that is mentioned under R¹, and U¹ represents —C₆H₄—O—CH₂-ω- or a group —(CH2)_{p′}-, whereby ω means the binding site to —CO—, and p′ is a whole number between 1 and 4,
   or of general formula IVc 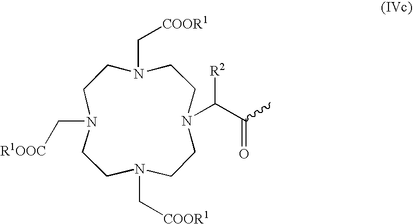
in which R¹ and R² have the above-mentioned meaning,
   or of general formula Vc A or Vc B 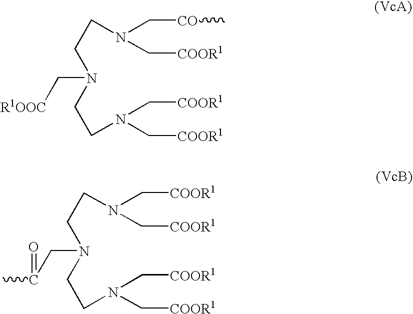
in which R¹ has the above-mentioned meaning,
   or of general formula VIc 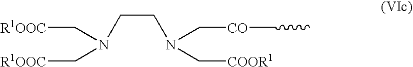
in which R¹ has the above-mentioned meaning,
   or of general formula VIIc 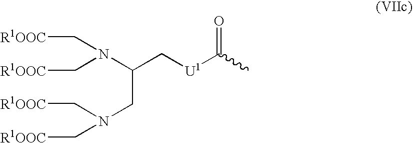
in which R¹ and U1 have the above-mentioned meaning, whereby ω means the binding site to —CO—
   or of general formula VIII 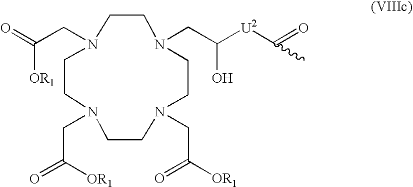
in which R¹ has the above-mentioned meaning,
and U² represents a straight-chain or branched, saturated or unsaturated C₁-C₂₀ alkylene group that optionally contains imino, phenylene, phenylenoxy, phenylenimino, amide, hydrazide, carbonyl, ester groups, oxygen, sulfur and/or nitrogen atoms and optionally is substituted by hydroxy, mercapto, oxo, thioxo, carboxy, carboxyalkyl, ester, and/or amino group(s),
and in radical K, optionally present free acid groups optionally can be present as salts of organic and/or inorganic bases or amino acids or amino acid amides, and L represents a radical that is selected from the radicals XXIIa) to XXIIc) below: 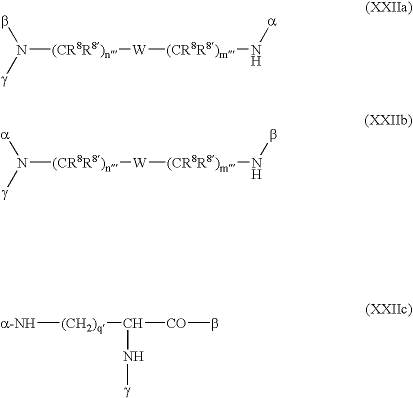
whereby n′″ and m′″, independently of one another, represent a whole number between 0 and 4, and m′″+n′″≧1; m′″+n′″ is preferably equal to 1, 2, or 3, and
R⁸ and R^{8′}, independently of one another, are either —H or —OH,
whereby in the case of m′″+n′″>1, any group —(CR⁸R^{8′})— can be different, and
W either is a direct bond, —O— or a phenylene group, which optionally can be substituted by 1 to 4 hydroxy groups,
and q′ is either 1, 2, 3 or 4,
whereby α means the binding site of L to the complex K, β is the binding site of L to the radical Q, and γ represents the binding site of L to the radical X, and
X stands for a group of formula (XXIII)
   ρ-Y—(CH₂)ₛ-(G)ₜ-(CH₂)_{s′}-ζ (XXIII)
whereby Y means a direct bond, a group —CO— or a group NR⁶, whereby
R¹ here stands for —H or a straight or branched, saturated or unsaturated C₁-C₁₅ carbon chain, which can be interrupted by 1-4 O atoms, 1-3 —NHCO groups, 1-3 —CONH groups, 1-2 —SO₂ groups, 1-2 sulfur atoms, 1-3 —NH groups or 1-2 phenylene groups,
   which optionally can be substituted with 1-2 OH groups, 1-2 NH₂ groups, 1-2 —COOH groups, or 1-2 —SO₃H groups,
   and which optionally is substituted with 1-10 OH groups, 1-5 —COOH groups, 1-2 —SO₃H groups, 1-5 NH₂ groups, or 1-5 C₁-C₄-alkoxy groups,
and G means either —O— or —SO₂—,
   s and s′, independently of one another, mean either 1 or 2, t means either 0 or 1, and
      ρ represents the binding site of X to L, and ζ represents the binding site of X to R_{f}.

In a preferred embodiment, R⁶ here is H or a C₁-C₆ alkyl group, which can be interrupted by 1-3 O atoms and which can be substituted with 1-4 —OH groups.

In an especially preferred embodiment, R⁶ here is a C₁-C₄ alkyl group.

In a preferred embodiment, G means the group —O—.

In an especially preferred embodiment, t=0.

In a preferred embodiment, W is a direct bond.

In a preferred embodiment, the radical R that is bonded to the linker L via a —CO—, —NR⁷— or a direct bond is a carbon chain with 1-30 C atoms that is interrupted by 1 to 10 oxygen atoms and/or is substituted with 1-10 OH groups.

In an especially preferred embodiment, R′ is a C1-C12 carbon chain that is bonded via a —CO—, —NR⁷— or direct bond to L that is interrupted by 1 to 6 oxygen atoms and/or is substituted with 1-6 OH groups.

Especially preferred compounds of general formula Ie are those with the macrocyclic compound K of general formula IIc.

The radical U in the metal complex K preferably means —CH₂— or C₆H₄—O—CH₂-ω, whereby ω stands for the binding site to —CO—.

In a preferred embodiment, U² is a C₁-C₆ alkylene chain, which optionally is interrupted by 1 to 2 —NHCO— groups and/or 1 to 2 O atoms, and which can be substituted with 1 to 3 —OH groups.

The radical U² in the metal complex K preferably means in particular
a linear alkylene group with 1 to 6 C atoms, in particular 2, 3 or 4 C atoms, or
a linear alkylene group with 1 to 6 C atoms, in particular 2, 3 or 4 C atoms, which is interrupted by 1 O atom, or
a linear alkylene group with 1 to 6 C atoms, in particular 2, 3 or 4 C atoms, which contains an —NHCO— group.

In an especially preferred embodiment, U² is an ethylene group.

The alkyl groups R² and R³ in the macrocyclic compound of general formula IIc can be straight-chain or branched. By way of example, methyl, ethyl, propyl, isopropyl, n-butyl, 1-methylpropyl, 2-methylpropyl, n-pentyl, 1-methylbutyl, 2-methylbutyl, 3-methylbutyl, and 1,2-dimethylpropyl can be mentioned. R² and R³, independently of one another, preferably mean hydrogen or C₁-C₄-alkyl.

In a quite especially preferred embodiment, R² stands for methyl and R³ stands for hydrogen.

The benzyl group or the phenyl group R² or R³ in the macrocyclic compound K of general formula IIc can also be substituted in the ring.

In another preferred embodiment of the invention, R′ means a monosaccharide radical with 5 or 6 C atoms, preferably glucose, mannose, galactose, ribose, arabinose or xylose or the deoxy sugars thereof, such as, for example, 6-deoxygalactose (fucose) or 6-deoxymannose (rhamnose) or peralkylated derivatives thereof. Glucose, mannose and galactose, in particular mannose, are especially preferred.

In another preferred embodiment of this invention, R′ is selected from one of the following radicals:
—C(O)CH₂O[(CH₂)₂O]ₚR′″
—C(O)CH₂OCH[CH₂OCH(CH₂OR′)₂]₂
—C(O)CH₂OCH₂CH[CH₂OCH(CH₂OR—)₂]₂
—R″N[(CH₂)₂O]ₚR′″
—N{[(CH₂)₂O]ₚR′″}₂
—R″NCH₂CH(OH)CH₂OH
—N[CH₂CH(OH)CH₂OH]₂
—R″NCH(CH₂OH)CH(OH)CH₂OH
—N[CH(CH₂OH)CH(OH)CH₂OH]₂
—R″NCH[CH₂OCH(CH₂OR′)₂]₂
—R″NCH₂CH[CH₂OCH(CH₂OR′)₂]₂
—R″NCH₂CH₂OCH[CH₂OCH(CH₂OR′)₂]₂
—R″NCH₂CH₂OCH₂CH[CH₂OCH(CH₂OR′)₂]₂
—N{CH[CH₂OCH(CH₂OR′″)₂]₂}₂
—N{CH₂CH[CH₂OCH(CH₂OR′″)₂]₂}₂
—R″NCH₂CH(OH)CH(OH)CH(OH)CH(OH)CH₂OH
—N[CH₂CH(OH)CH(OH)CH(OH)CH(OH)CH₂OH]₂
and a complex of formula (IIc), with Q in the meaning of a direct bond,

whereby R¹, R², R³ and U are as defined above for formula (IIc),

p is either 1, 2, 3, 4, 5, 6, 7, 8 or 9,

R′″ is either H or CH₃, and R″ is either H or a C1 to C₄ alkyl radical.

p is preferably 1, 2, 3, or 4.

In a quite especially preferred embodiment, R′ is a radical, bonded via —CO— to L, of the formula: —C(O)CH₂O[(CH₂)₂O]ₚR′″.

With p and R′″ in the above-indicated meaning, especially preferred R′″ is the group CH₃.

In another preferred embodiment, Q has the meaning of a group that is selected from:
δ-CO—(CH₂)_{n″}-ε

whereby

n″ is a whole number from 1 to 5, and

L simultaneously has the meaning of a group of formula XXIIa or XXIIXb.

In another preferred embodiment, Q has the meaning of a group that is selected from:
δ-NH—(CH₂)_{n″}-ε

whereby

n″ is a whole number from 1 to 5, and

L simultaneously has the meaning of a group XXIIc.

Of the compounds of general formula Ie according to the invention, in addition those are preferred in which R_{f} means —CₙF₂ₙ₊₁; i.e., E in the formula —CₙF₂ₙE means a fluorine atom. n preferably stands for the numbers 4-15. The radicals —C₄F₉, —C₆F₁₃, —C₈F₁₇, —C₁₂F₂₅ and —C₁₄F₂₉ are quite especially preferred.

The nitrogen-containing radical L in the general formula Ie, which represents the “skeleton,” means the amino acid radical (XXIIc) in a preferred embodiment of the invention.

In another preferred embodiment, the nitrogen-containing radical L in general formula I represents a diamine radical of formula (XXIIb) or (XXIIa).

The following are used as quite especially preferred compounds of general formula Ie:
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-(1-O-α-d-mannopyranosyl)-acetamide, Gd complex
1H,1H,2H,2H,4H,4H,5H,5H-3-N-[1,4,7-Tris-(carboxylatomethyl)-1,4,7,10-tetraazacyclododecane-10-N-(pentanoyl-3-aza-4-oxo-5-methyl-5-yl)]-perfluorotridecyl-N-2-(1-O-α-d-mannopyranosyl)-acetamide, Gd complex
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-[1,3-bis-(2-hydroxy-1-hydroxymethyl-ethoxy)-prop-2-oxy]-acetamide, Gd complex
1,4,7-{Tris(carboxylatomethyl)-10-[(3-aza-4-oxo-hexan-5-ylic)-acid-N-1H,1H,2H,2H,4H,4H,5H,5H-3-N-[1,4,7-tris-(carboxylatomethyl)-1,4,7,10-tetraazacyclododecane-10-N-(pentanoyl-3-aza-4-oxo-5-methyl-5-yl)]-perfluorotridecylamide}-1,4,7,10-tetraazacyclododecane, Gd complex
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-[2-(2-methoxyethoxy)-ethoxy]-acetamide, Gd complex
6-N-[1,4,7-Tris-(carboxylatomethyl)-1,4,7,10-tetraazacyclododecane-10-N-(pentanoyl-3-aza-4-oxo-5-methyl-5-yl)]-2-N-(2H,2H,4H,4H,5H,5H-3-oxaperfluorotridecanoyl)-L-lysine-[2-{1-O-α-d-mannopyranosyl)-ethyl]-amide, Gd complex
6-N-[1,4,7-Tris-(carboxylatomethyl)-1,4,7,10-tetraazacyclododecane-10-N-(pentanoyl-3-aza-4-oxo-5-methyl-5-yl)]-2-N-(2H,2H,4H,4H,5H,5H-3-oxaperfluorotridecanoyl)-L-lysine-{[N-(2S,3R,4R,5R),-2,3,4,5,6-pentahydroxyhexyl]-N-methyl}-amide, Gd complex
6-N-[1,4,7-Tris-(carboxylatomethyl)-1,4,7,10-tetraazacyclododecane-10-N-(pentanoyl-3-aza-4-oxo-5-methyl-5-yl)]-2-N-(2H,2H,4H,4H,5H,5H-3-oxaperfluorotridecanoyl)-L-lysine-(2-{2-[2-(2-methoxyethoxy)-ethoxy]-ethoxy}-ethyl)-amide, Gd complex
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-(2-{2-[2-(2-methoxyethoxy)-ethoxy]-ethoxy}-ethoxy)-acetamide, Gd complex
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-{-2-[2-(2-methoxyethoxy)-ethoxy]-ethoxy}-acetamide, Gd complex
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-(2-methoxyethoxy)-acetamide, Gd complex.

The metal complex
N-{[1,4,7-Tris-(carboxylatomethyl)-1,4,7,10-tetraazacyclododecane-10-N-(pentanoyl-3-aza-4-oxo-5-methyl-5-yl)]-2-aminoethyl}-N-(1H,1H,2H,2H-perfluorodecyl)-2-{-2-[2-(2-methoxyethoxy)-ethoxy]-ethoxy}-acetamide, Gd complex,
   is most preferably used, in particular as a contrast medium in MR imaging.

In this case, these are known compounds that are described in the unpublished DE102005033902.6-44. Also, their production can be found in this application. It has been shown, surprisingly enough, that these compounds are also very well suited as contrast media, in particular MRI contrast media, for diagnosis of Alzheimer's disease.

In another preferred embodiment of the invention, the metal complexes of general formula If

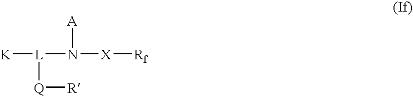

can be used,

whereby
R′ either
   represents a monosaccharide or oligosaccharide radical bonded via the 1-OH, which is optionally peralkylated,
   in this case Q has the meaning of a group that is
   selected from:
      δ-CO—(CH₂)_{n″}-ε
      δ-NH—(CH₂)_{n″}-ε
      δ-(CH₂)_{m″}-ε

whereby
n″ is a whole number from 1 to 5, and
m″ is a whole number from 1 to 6, and
whereby δ indicates the binding site to the linker L, and ε the binding site to the radical R′;
or
R′ has one of the following meanings, then Q has the meaning of a direct bond: R′ means a polar radical that is selected from
   the complexes K of general formulas IIc to VIII′c, whereby R¹ here means a hydrogen atom or a metal ion equivalent of the atomic numbers 20-29, 31-33, 37-39, 42-44, 49 or 57-83, and the radicals R², R³, R⁴, U and U¹ have the meanings that are indicated below,
      or
   a carbon chain with 1-30 C atoms that is bonded via —CO—, —NR⁶— or a direct bond to the linker L,
      which can be straight or branched, saturated or unsaturated,
and which optionally is interrupted by 1-10 oxygen atoms, 1-5 —NHCO— groups, 1-5 —CONH groups, 1-2 sulfur atoms, 1-5 —NH groups, or 1-2 phenylene groups, which optionally can be substituted with 1-2 OH groups, 1-2 NH₂ groups, 1-2 —COOH groups, or 1-2 —SO₃H groups,
and which optionally is substituted with 1-10 OH groups, 1-5 —COOH groups, 1-2 SO₃H groups, 1-5 NH₂ groups, or 1-5 C₁-C₄-alkoxy groups, whereby R⁶ means H or C₁-C₄ alkyl,
R_{f} means a perfluorinated, straight-chain or branched carbon chain with the formula —CₙF₂ₙE, in which E represents a terminal fluorine atom, chlorine atom, bromine atom, iodine atom or hydrogen atom, and n stands for the numbers 4-30,
X stands for a group of formula (XXIII)
   ρ-Y—(CH₂)ₛ-(G)_{t-(CH}₂)_{s′}-ζ (XXIII)

and G means either —O— or —SO₂—,
s and s′, independently of one another, mean either 1 or 2, t means either 0 or 1, and
   ρ means the binding site of X to L, and ζ the binding site of X to R_{f}.
K stands for a metal complex of general formula IIc,

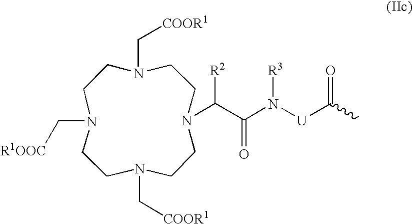

in which
R¹ means a hydrogen atom or a metal ion equivalent of atomic numbers 21-29, 31-33, 37-39, 42-44, 49 or 57-83,
   provided that at least two R¹ stand for metal ion equivalents,
R² and R³, independently of one another, represent hydrogen, C₁-C₇-alkyl, benzyl, phenyl, —CH₂OH or —CH₂OCH₃, and
U represents —C₆H₄—O—CH₂-ω-, —(CH₂)₁₋₅-ω, a phenylene group, —CH₂—NHCO—CH₂—CH(CH₂COOH)—C₆H₄-ω-, —C₆H₄—(OCH₂CH₂)₀₋₁-N(CH₂COOH)—CH₂-ω or a C₁-C₁₂-alkylene group or a —(CH₂)₇₋₁₂—C₆H₄—O group that optionally is interrupted by one or more oxygen atoms, 1 to 3 —NHCO groups or 1 to 3 —CONH groups and/or substituted with 1 to 3 —(CH₂)₀₋₅COOH groups,
   whereby ω stands for the binding site to —CO—,
or
   of general formula IIIc 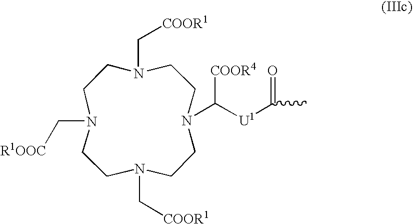
in which R¹ has the above-mentioned meaning, R⁴ represents hydrogen or a metal ion equivalent that is mentioned under R¹, and U¹ represents —C₆H₄—O—CH₂-ω- or a group —(CH₂)_{p′}—, whereby ω means the binding site to —CO—, and p is a whole number between 1 and 4,
   or of general formula IVc 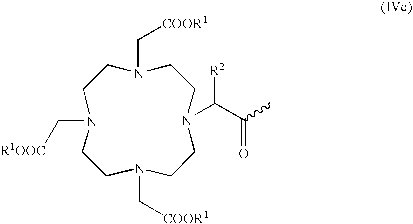
in which R¹ and R² have the above-mentioned meaning,
   or of general formula VcA or VcB 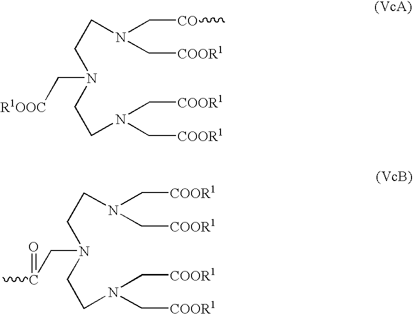
in which R¹ has the above-mentioned meaning,
   or of general formula VIc 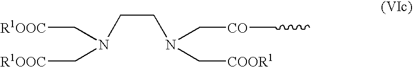
in which R¹ has the above-mentioned meaning,
   or of general formula VIIc 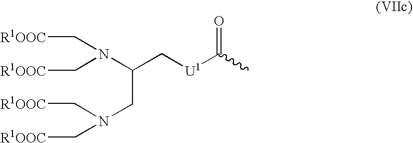
in which R¹ and U¹ have the above-mentioned meaning, whereby o) means the binding site to —CO—,
   or of general formula VIIIc

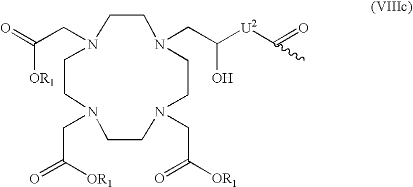

in which R¹ has the above-mentioned meaning,

and U² represents a straight-chain or branched, saturated or unsaturated C₁-C₂₀ alkylene group that optionally contains imino groups, phenylene groups, phenylenoxy groups, phenylenimino groups, amide groups, hydrazide groups, carbonyl groups, ester groups, oxygen atoms, sulfur atoms and/or nitrogen atoms and optionally is substituted by hydroxy groups, mercapto groups, oxo groups, thioxo groups, carboxy groups, carboxyalkyl groups, ester groups and/or amino groups,
or of general formula VIII′c 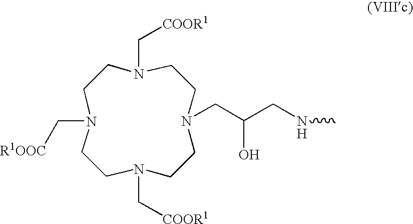
in which R¹ has the above-mentioned meaning,
and in the radical K, optionally present free acid groups optionally can be present as salts of organic and/or inorganic bases or amino acids or amino acid amides, and
L represents a radical that is selected from the radicals XXIVa) to XXIVg) below: 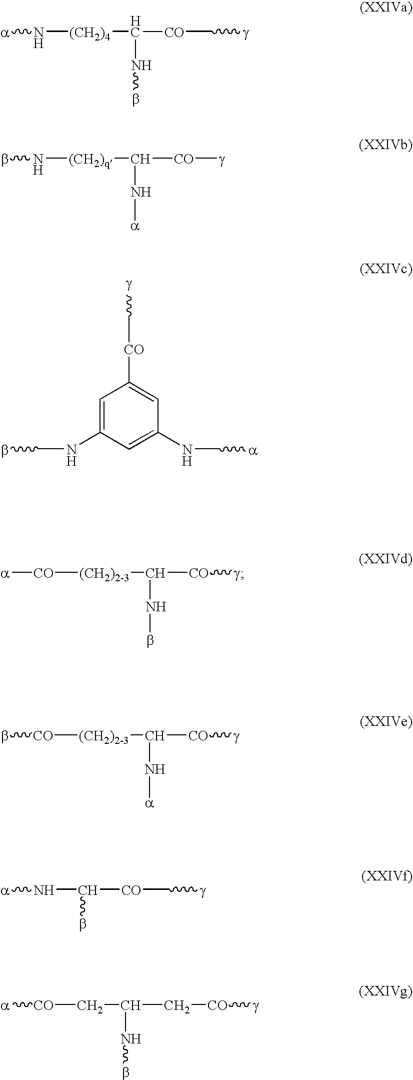
whereby q′ is either 1, 2, 3 or 4, and
whereby α means the binding site of L to the complex K, β is the binding site of L to the radical Q, and γ represents the binding site of L to N of formula (If), and
A stands for a straight or branched, saturated or unsaturated C₁-C₁₅ carbon chain, which can be interrupted by 1-4 O atoms, 1-3 —NHCO— groups, 1-3 —CONH— groups, 1-2 —SO₂— groups, 1-2 sulfur atoms, 1-3 —NH— groups or 1-2 phenylene groups, which optionally can be substituted with 1-2 —OH groups, 1-2 —NH₂ groups, 1-2 —COOH groups, or 1-2 —SO₃H groups,
   and which optionally is substituted with 1-10 —OH groups, 1-5 —COOH groups, 1-2 —SO₃H groups, 1-5 —NH₂ groups, or 1-5 C₁-C₄-alkoxy groups.

In a preferred embodiment, A is a radical
—(CH₂)_{s″}—(O)ₜ—(CH₂)_{s′″}-Z
whereby s″ represents a whole number between 1 and 4,

s′″ represents a whole number between 0 and 4,

t′ is 0 or 1, and

Z is either —H, —OH, or —COOH.

In a preferred embodiment, G means the group —O—.

In another preferred embodiment, Q has the meaning of a group
δ-CO—(CH₂)_{n′}-ε

whereby

n″ is a whole number from 1 to 5, n″ is preferably equal to 1, 2 or 3.

In a preferred embodiment, the radical R′ that is bonded to the linker L via —CO—, —NR⁷— or a direct bond (Q is a direct bond) is a carbon chain with 1-30 C atoms that is interrupted by 1 to 10 oxygen atoms and/or is substituted with 1-10 —OH groups. In an especially preferred embodiment, R′ is a C₁-C₁₅ carbon chain that is bonded via —CO—, —NR⁷— or a direct bond to L that is interrupted by 1 to 8 oxygen atoms and/or is substituted with 1-8 OH groups.

In an especially preferred embodiment of this invention, R′ is selected from one of the following radicals:
—C(O)CH₂O[(CH₂)₂O]ₚR′″
—C(O)CH₂OCH[CH₂OCH(CH₂OR′″)₂]₂
—C(O)CH₂OCH₂CH[CH₂OCH(CH₂OR′)₂]₂
—R″N[(CH2)₂O]ₚR′″
—N{[(CH₂)₂O]ₚR′″}₂
—R″NCH₂CH(OH)CH₂OH
—N[CH₂CH(OH)CH₂OH]₂
—R″NCH(CH₂OH)CH(OH)CH₂OH
—N[CH(CH₂OH)CH(OH)CH₂OH]₂
—R″NCH[CH₂OCH(CH₂OR′″)₂]₂
—R″NCH₂CH[CH₂OCH(CH₂OR′″)₂]₂
—R″NCH₂CH₂OCH[CH₂OCH(CH₂OR′″)₂]₂
—R″NCH₂CH₂OCH₂CH[CH₂OCH(CH₂OR′″)₂]₂
—N{CH[CH₂OCH(CH₂OR′″)₂]₂}₂
—N{CH₂CH[CH₂OCH(CH₂OR′″)₂]₂}₂
—R″NCH₂CH(OH)CH(OH)CH(OH)CH(OH)CH₂OH
—N[CH₂CH(OH)CH(OH)CH(OH)CH(OH)CH₂OH]₂

and a complex of formula (IIc), with Q in the meaning of a direct bond,

whereby R¹, R², R³ and U are defined as above for formula (IIc),

p is either 1, 2, 3, 4, 5, 6, 7, 8 or 9,

R′″ is either H or CH₃, and R″ is either H or a C₁ to C₄-alkyl radical.

p is preferably 1, 2, 3, or 4.

In a quite especially preferred embodiment, R′ is a radical, bonded to L via —CO—, of the formula: —C(O)CH₂O[(CH₂)₂O]ₚR′″

with p and R′″ in the above-indicated meaning; R′″ is especially preferably the group CH₃.

Especially preferred compounds of general formula If are those with the macrocyclic compound K of general formula IIc.

The radical U in the metal complex K preferably means —CH₂— or C₆H₄—O—CH₂-ω, whereby ω stands for the binding site to —CO—.

In a preferred embodiment, U² is a C₁-C₆ alkylene chain, which optionally is interrupted by 1 to 2 —NHCO— groups and/or 1 to 2 O atoms, and which can be substituted with 1 to 3 —OH groups.

The radical U² in the metal complex K preferably means
a linear alkylene group with 1 to 6 C atoms, in particular 2, 3 or 4 C atoms, or
a linear alkylene group with 1 to 6 C atoms, in particular 2, 3 or 4 C atoms, which is interrupted by 1 O atom, or
a linear alkylene group with 1 to 6 C atoms, in particular 2, 3 or 4 C atoms, which contains an —NHCO group.

In an especially preferred embodiment, U² is an ethylene group.

The alkyl groups R² and R³ in the macrocyclic compound of general formula IIc can be straight-chain or branched. By way of example, methyl, ethyl, propyl, isopropyl, n-butyl, 1-methylpropyl, 2-methylpropyl, n-pentyl, 1-methylbutyl, 2-methylbutyl, 3-methylbutyl, and 1,2-dimethylpropyl can be mentioned. R² and R³, independently of one another, preferably mean hydrogen or C₁-C₄-alkyl.

In a quite especially preferred embodiment, R² stands for methyl and R³ stands for hydrogen.

In a preferred embodiment of the invention, R′ means a monosaccharide radical with 5 or 6 C atoms, preferably glucose, mannose, galactose, ribose, arabinose or xylose or the deoxy sugars thereof, such as, for example, 6-deoxygalactose (fucose) or 6-deoxymannose (rhamnose) or peralkylated derivatives thereof. Glucose, mannose and galactose, and peralkylated derivatives thereof, in particular mannose and peralkylated mannose, are especially preferred.

Peralkylated monosaccharides or oligosaccharides can be alkylated with identical or different linear or branched C₁-C₆-alkyl groups; they are preferably permethylated.

In another preferred embodiment of this invention, R′ is selected from
a carbon chain with 1-15 C atoms that is bonded via —CO—, —NR⁶— or a direct bond to the linker L,
which can be straight or branched, saturated or unsaturated, and which optionally is interrupted by 1-10 oxygen atoms, and which optionally is substituted with 1-10 —OH groups,
whereby R⁶ means H or C₁-C₄ alkyl,
and a complex of formula (IIc),
   whereby R¹, R², R³ and U are defined as above for formula (IIc). Of the compounds of general formula If according to the invention, in addition those are preferred in which R_{f} means —CₙF₂ₙ₊₁; i.e., E in the formula —CₙF₂ₙE means a fluorine atom. n preferably stands for the numbers 4-15. The radicals —C₄F₉, —C₆F₁₃, —C₈F₁₇, —C₁₂F₂₅ and —C₁₄F₂₉ are quite especially preferred.

The radical L in general formula If, which represents the “skeleton,” means an amino acid radical (XXIVa) or (XXIVb) in a preferred embodiment of the invention.

In another preferred embodiment, the radical L in the general formula I represents a radical of the formulas (XXIVc), (XXIVd), (XXIVe) or (XXIVf).

The following are used as quite especially preferred compounds of general formula If:

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-[(1H,1H,2H,2H-perfluorodecyl)-methyl]-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-[(1H,1H,2H,2H-perfluorododecyl)-methyl]-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-[(1H,1H,2H,2H-perfluorooctyl)-methyl]-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-[(1H,1H,2H,2H-perfluoro-9-methyldecyl)-methyl]-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-[(1H,1H,2H,2H,3H,3H-perfluoroundecyl)-methyl]-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-[(1H,1H,2H,2H,4H,4H,5H,5H-3-oxa-perfluorotridecyl)-methyl]-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-N-(2-methoxyethyl)-N-(1H,1H,2H,2H,4H,4H,5H,5H-3-oxa-perfluorotridecyl)-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-(1-O-α-d-carbonylmethylmanno-pyranose)-L-lysine-N-(2-methoxyethyl)-N-(1H,1H,2H,2H-perfluorodecyl)-amide, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N-[(3-hydroxy-2,2-dihydroxymethyl-propoxy)-acetyl]-L-lysine-[(1H,1H,2H,2H-perfluorodecyl)-methyl]-amid, Gd complex

6-N-[1,4,7-Tris-(carboxylatomethyl)-1,4,7,10-tetraazacyclododecane-10-N-(pentanoyl-3-aza-4-oxo-5-methyl-5-yl)]-2-N- [1-O-α-d-carbonylmethyl-(2,3,4,6-tetra-O-methyl)mannopyranose]-L-lysine-[(1H,1H,2H,2H-perfluorodecyl)-methyl]-amide, Gd complex

2,6-N,N-Bis-[1,4,7-tris-(carboxylatomethyl)-1,4,7,10-tetraazacyclododecane-10-N-(pentanoyl-3-aza-4-oxo-5-methyl-5-yl)]-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-(1-O-α-d-carbonylmethylmanno-pyranose)-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-(2-hydroxyacetyl)-L-lysine-[(1H,1H,2H,2H-perfluorodecyl)-methyl]-amide, Gd complex

—2-N-[1,4,7-Tris-(carboxylatomethyl)-1,4,7,10-tetraazacyclododecane-10-N-(pentanoyl-3-aza-4-oxo-5-methyl-5-yl)]-6-N-(2-methoxyacetyl)-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-[2-(2-methoxyethoxy)-acetyl]-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-{2-[2-(2-methoxyethoxy)-ethoxy]-acetyl}-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-(2-{2-[2-(2-hydroxyethoxy)-ethoxy]-ethoxy}-acetyl)-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-(2-{2-[2-(2-methoxyethoxy)-ethoxy]-ethoxy}-acetyl)-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-[2-(2-hydroxyethoxy)-acetyl]-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-{2-[2-(2-hydroxyethoxy)-ethoxy]-acetyl}-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-[2-(2-{2-[2-(2-methoxyethoxy)-ethoxy]-ethoxy}-ethoxy)-acetyl]-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-[2-(2-{2-[2-(2-hydroxyethoxy)-ethoxy]-ethoxy}-ethoxy)-acetyl]-L-lysine-[(1H,1H,2H,2H-perfluorodecyl)-methyl]-amide, Gd complex

2-N-[1,4,7-Tris-(carboxylatomethyl)-1,4,7,10-tetraazacyclododecane-10-N-(pentanoyl-3-aza-4-oxo-5-methyl-5-yl)]-6-N-{[1,3-bis-(2-benzyloxy-1-benzyloxymethyl-ethoxy)-prop-2-yl]-acetyl}-L-lysine-[(1H,1H,2H,2H-perfluorodecyl)-methyl]-amide, Gd complex.

In this case, these are known compounds that are described in the unpublished DE 102005033903.4. Also, their production can be found in this application. It has been shown, surprisingly enough, that these compounds are also very well suited as contrast media, in particular MRI contrast media, for diagnosis of Alzheimer's disease.

In another preferred embodiment of the invention, galenical formulations that contain paramagnetic and diamagnetic perfluoroalkyl-containing substances can be used. The paramagnetic and diamagnetic substances are preferably present dissolved in an aqueous solvent.

All above-mentioned metal complexes that contain a paramagnetic metal ion can be used as paramagnetic perfluoroalkyl-containing compounds in the formulations according to the invention.

The diamagnetic perfluoroalkyl-containing substances are those of general formula XX (see WO 02/13874):
R^{F}-L²-B² (XX)
in which R^{F} represents a straight-chain or branched perfluoroalkyl radical with 4 to 30 carbon atoms, L² stands for a linker, and B² stands for a hydrophilic group. The linker L² is preferably a direct bond, an —SO₂ group, or a straight-chain or branched carbon chain with up to 20 carbon atoms, which can be substituted with one or more —OH, —COO⁻, or —SO₃ groups, and/or optionally contains one or more —O—, —S—, —CO—, —CONH—, —NHCO—, —CONR⁹—, —NR⁹CO—, —SO₂—, —PO₄⁻—, —NH—, or —NR⁹ groups, an aryl ring or a piperazine, whereby R⁹ stands for a C₁- to C₂₀-alkyl radical, which in turn can contain one or more O atoms, and/or can be substituted with —COO⁻ or SO₃ groups.

The hydrophilic group B² is preferably a monosaccharide or disaccharide, which contains one or more adjacent —COO⁻ or —SO₃⁻ groups, a dicarboxylic acid, an isophthalic acid, a picolinic acid, a benzenesulfonic acid, a tetrahydropyrandicarboxylic acid, a 2,6-pyridinedicarboxylic acid, a quarternary ammonium ion, an amino-polycarboxylic acid, an aminodipolyethylene glycolsulfonic acid, an aminopolyethylene glycol group, an SO₂—(CH₂)₂—OH group, a polyhydroxyalkyl chain with at least two hydroxyl groups or one or more polyethylene glycol chains with at least two glycol units, whereby the polyethylene glycol chains are terminated by an —OH group or an —OCH₃ group.

Additional suitable diamagnetic perfluoroalkyl-containing compounds are conjugates that consist of cyclodextrin and perfluoroalkyl-containing compounds. These conjugates consist of α-, β- oder γ-cyclodextrin and compounds of general formula XXII (see WO 02/13874)
A¹-L³-R^{F} (XXII)
in which A¹ stands for an adamantan molecule, a biphenyl molecule or an anthracene molecule, L³ stands for a linker, and R^{F} stands for a straight-chain or branched perfluoroalkyl radical with 4 to 30 carbon atoms. The linker L³ is a straight-chain hydrocarbon chain with 1 to 20 carbon atoms, which can be interrupted by one or more oxygen atoms, one or more CO—, SO₂—, CONH—, NHCO—, CONR—, NRCO—, NH—, NR groups or a piperazine, whereby R is a C₁-C₅-alkyl radical.

In another preferred embodiment, those substances of general formula XXI can be used as diamagnetic perfluoroalkyl-containing substances:
R^{F}—X¹ (XXI)
in which R^{F} represents a straight-chain or branched perfluoroalkyl radical with 4 to 30 carbon atoms, and X¹ is a radical that is selected from the group of the following radicals (in this case, n is a number between 1 and 10):

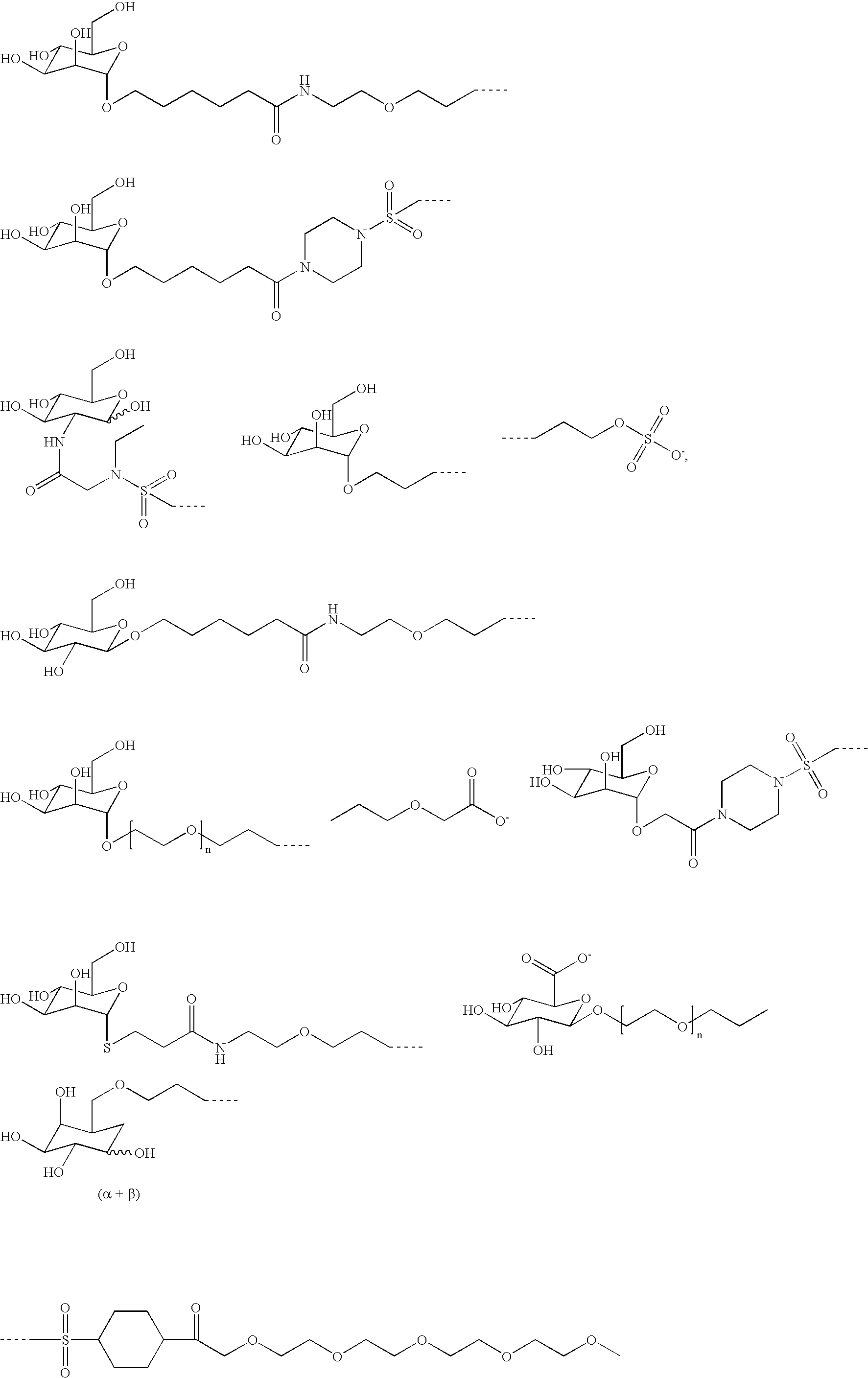

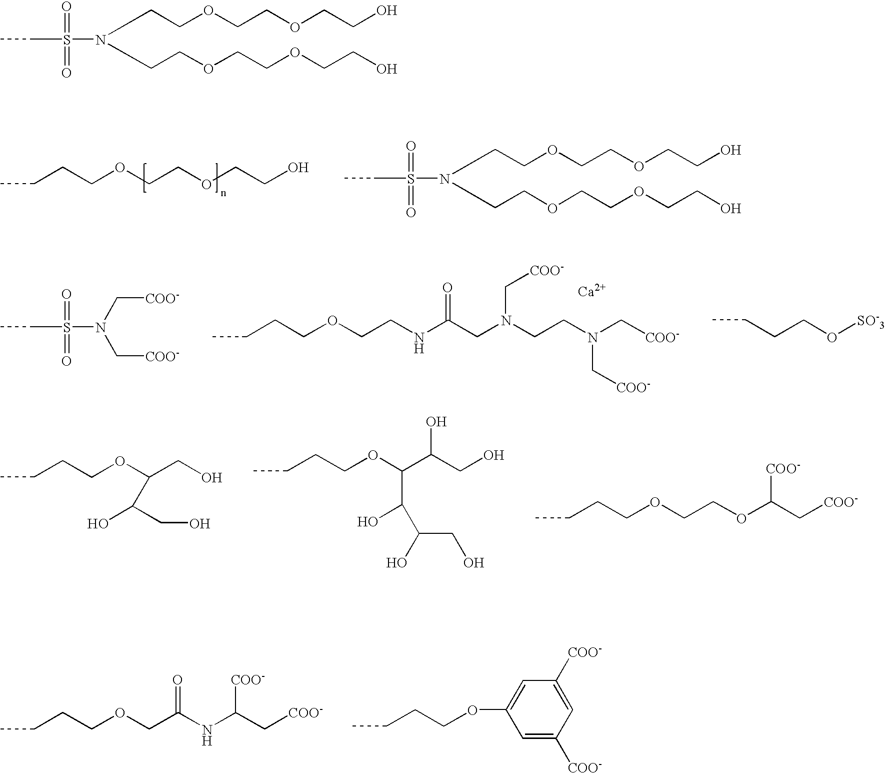

In R¹ of the above-described metal complexes, optionally present acid hydrogen atoms, i.e., those that had not been substituted by the central ion, can be replaced optionally completely or partially by cations of inorganic and/or organic bases or amino acids or amino acid amides in all described metal complexes that can be used according to the invention.

The term “metal ion equivalent” as used in the application is a conventional term that is known to one skilled in the art in the area of complex chemistry. A metal ion equivalent is an equivalent of metal ions that can bind to a, e.g., carboxylate group instead of hydrogen. For example, a Gd³⁺ can bind to 3 carboxylate groups, i.e., ⅓ Gd³⁺ corresponds to the metal ion equivalent of R¹ in formula (IIb) or (IIc) if the metal is gadolinium.

The compounds that can be used according to the invention are especially preferably intended for use in ¹H-MR imaging. Therefore, the metal ion of the signaling group must in this case be paramagnetic. These are in particular the divalent and trivalent ions of elements of the atomic numbers 21-29, 42, 44 and 58-70. Suitable ions are, for example, chromium(II), iron(II), cobalt(II), nickel(II), copper(II), praseodymium(III), neodymium(III), samarium(III) and ytterbium(III) ions. Because of their strong magnetic moments, gadolinium(III), terbium(III), dysprosium(III), holmium(III), erbium(III), iron(III) and manganese(II) ions are especially preferred.

For the following tests, the gadolinium complexes were used, since the gadolinium of all paramagnetic ions has the greatest influence on the signal amplification in the MRI.

The use of metal complexes that contain gadolinium as a contrast element for use in MR imaging is therefore quite especially preferred.

In summary, it has been determined that the gadolinium complexes MK 1-28 that are cited as quite especially preferred compounds in Table 1 meet the criteria according to the invention. The physical parameters of these metal complexes are cited in Table 1.

In another embodiment of this invention, metal complexes as described above that contain radioisotopes are used: for the use of the compounds according to the invention in radiodiagnosis, the metal ion must be radioactive. For example, radioisotopes of elements with the atomic numbers 27, 29, 31-33, 37-39, 43, 49, 62, 64, 70, 75 and77 are suitable. Technetium, gallium, indium, rhenium and yttrium are preferred. Such radioisotopes are signaling groups in terms of this invention.

The agents according to the invention are also suitable as radiodiagnostic agents based on their advantageous radioactive properties and the good stability of the complex compounds contained therein. Details of such application and dosage are described in, e.g., “Radiotracers for Medical Applications,” CRC Press, Boca Raton, Fla.

The compounds and agents according to the invention can also be used in positron-emission tomography, which uses positron-emitting isotopes, such as, e.g., ⁴³Sc, ⁴⁴Sc, ⁵²Fe, ⁵⁵Co, ⁶⁸Ga and ⁸⁶Y (Heiss, W. D.; Phelps, M. E.; Positron Emission Tomography of Brain, Springer Verlag Berlin, Heidelberg, New York 1983).

In another embodiment according to the invention, the metal complexes are labeled with ¹⁸F and/or ¹¹C as signaling groups and thus can be used as PET tracers in positron emission tomography.

If the compound according to the invention is intended for use in x-ray diagnosis, the metal ion is preferably derived from an element of a higher atomic number to achieve sufficient absorption of the x-ray beams. Diagnostic agents with metal ions of elements of atomic numbers 25, 26 and 39 as well as 57-83 are suitable.

Manganese(II), iron(II), iron(III), praseodymium(III), neodymium(III), samarium(III), gadolinium(III), ytterbium(III) or bismuth(III) ions, in particular dysprosium(III) ions and yttrium(III) ions, are preferred.

The agents according to the invention are extremely well suited as x-ray contrast media, whereby it is especially to be emphasized that with them, no signs of the anaphylaxis-like reactions that are known from the iodine-containing contrast media can be detected in biochemical-pharmacological studies. They are especially valuable owing to their advantageous absorption properties in ranges of higher tube voltages for digital subtraction techniques.

If the compound according to the invention is intended for use in the single photon emission computer tomography (SPECT), the metal complexes that can be used according to the invention are also radiolabeled. Suitable radionuclides are gamma-emitters, such as technetium-99 or iodine-123. In this case, these radionuclides represent a signaling group in terms of this invention.

In general, the agents according to the invention are dosed for use as x-ray contrast media analogously to, for example, meglumine-diatrizoate in amounts of 0.1-5 mmol/kg, preferably 0.25-1 mmol/kg.

Suitable inorganic cations are, for example, the lithium ion, the potassium ion, the calcium ion, and in particular the sodium ion. Suitable cations of organic bases are, i.a., those of primary, secondary or tertiary amines, such as, for example, ethanolamine, diethanolamine, morpholine, glucamine, N,N-dimethylglucamine and in particular N-methylglucamine. Suitable cations of amino acids are, for example, those of lysine, arginine and ornithine, as well as the amides of otherwise acidic or neutral amino acids.

Usually, pharmaceutical agents are administered that contain at least one physiologically compatible compound that can be used according to the invention, optionally with the additives that are commonly used in galenicals.

The production of the pharmaceutical agents according to the invention is carried out in a way that is known in the art by the complex compounds according to the invention—optionally with the addition of the additives that are commonly used in galenicals—being suspended or dissolved in aqueous medium and then the suspension or solution optionally being sterilized. Suitable additives are, for example, physiologically harmless buffers (such as, for example, tromethamine), additives of complexing agents or weak complexes (such as, for example, diethylenetriaminepentaacetic acid or the Ca complexes that correspond to the metal complexes according to the invention) or—if necessary—electrolytes, such as, for example, sodium chloride, or—if necessary—antioxidants, such as, for example, ascorbic acid.

If suspensions or solutions of the agents according to the invention in water or physiological salt solution are desired for enteral or parenteral administration or other purposes, they are mixed with one or more adjuvant(s) that are commonly used in galenicals [for example, methyl cellulose, lactose, mannitol] and/or surfactant(s) [for example, lecithins, Tween®, Myrj®] and/or flavoring substance(s) for taste correction [for example, ethereal oils].

In principle, it is also possible to produce the pharmaceutical agents according to the invention without isolating the complexes. In any case, special care must be used to perform the chelation so that the complexes according to the invention are virtually free of non-complexed metal ions that have a toxic action.

This can be ensured, for example, using color indicators such as xylenol orange by control titrations during the production process. The invention therefore also relates to processes for the production of complex compounds and salts thereof. As a final precaution, there is a purification of the isolated complex.

In the in-vivo application of the agents according to the invention, the latter can be administered together with a suitable vehicle, such as, for example, serum or physiological common salt solution and together with another protein, such as, for example, human serum albumin (HSA).

The agents according to the invention are usually administered parenterally, preferably i.v.

The pharmaceutical agents according to the invention preferably contain 0.1 μmol-2 mol/l of the complex and are generally dosed in amounts of 0.0001-5 mmol/kg of body weight.

The substances that can be used according to the invention show, on the one hand, the high effectiveness in detecting amyloid plaques that is necessary to load the body with the lowest possible amounts of foreign substances and at the same time to make a reliable diagnosis and, on the other hand, an excellent compatibility. Another advantage is the non-invasive nature of the studies, by which the stress on the patient is minimized.

The good water solubility and low osmolality of the agents according to the invention allows the production of highly concentrated solutions to keep the volume burden of the circulatory system within reasonable limits and to compensate the dilution by the bodily fluid. In addition, the agents according to the invention have a high stability in vivo, so that a release or an exchange of the ions bonded in the complexes takes place only extremely slowly within the time in which the new contrast media are completely excreted again.

In general, the agents according to the invention are dosed for use as MR diagnostic agents in amounts of 0.0001-5 mmol/kg of body weight, preferably 0.005-0.5 mmol/kg.

In the in-vivo application of the agents according to the invention, the latter can be administered together with a suitable vehicle, such as, for example, serum, or physiological common salt solution and together with another protein, such as, for example, human serum albumin. The dosage is in this case dependent on the type of cellular disorder, the metal ion that is used, and the type of imaging method.

The agents according to the invention are usually administered parenterally, preferably i.v. They can, however, also be administered via other methods of administration, e.g., intraarterially, intracutaneously, subcutaneously, percutaneously, intramuscularly, locally, by inhalation, rectally, enterally (perorally), etc.

In particular, the metal complexes that can be used according to the invention and the formulations that can be used according to the invention that consist of paramagnetic and diamagnetic perfluoroalkyl-containing substances that can be used for 1H-MRI imaging and/or x-ray diagnosis and/or another imaging process are extremely well suited as contrast media for diagnosis of amyloid-containing plaques both in vitro and in vivo, preferably in vivo.

In addition, they are suitable for use for the production of a diagnostic agent for visualizing amyloid-containing plaques.

In a preferred embodiment, they can be used for the diagnosis of Alzheimer's disease.

In another preferred embodiment, they can be used for differentiating between Alzheimer's disease and other dementia diseases.

In a preferred embodiment, they can be used for early detection of Alzheimer's disease.

In a preferred embodiment, they can be used for monitoring the course of treatment in treated Alzheimer patients, in particular in whom the loading of the brain with amyloid plaques before the beginning of treatment and/or during the treatment and/or after the completion of treatment is determined by using metal complexes in MR imaging, in x-ray diagnosis or by another imaging process. In this case, the following steps are performed:
I) Administration of a diagnostically effective amount of at least one metal complex, usable according to the invention, to an Alzheimer patient
II) Recording of an MR image, an x-ray image or an image with application of another imaging process
III) At least one repetition of steps I) and II) at another point in time
IV) Comparison of the images obtained
   whereby the Alzheimer patient is subjected to a treatment of his disease and this treatment can begin before or after the first image is recorded.

It is suggested that amyloid plaques have already formed in humans who still do not have any detectable symptoms of Alzheimer's disease. Such humans have an increased risk of contracting Alzheimer's disease at an advanced stage. The metal complexes that can be used according to the invention can therefore be used as contrast media, in particular for MRT, for identifying patients at risk for developing Alzheimer's disease.

In addition, this invention relates to a process for diagnosis of Alzheimer's disease that comprises the steps
I) Administration of a diagnostically effective amount of at least one complex according to the invention to an Alzheimer patient
II) Recording of an MR image, an x-ray image or an image with use of another imaging process
III) Optionally at least one repetition of steps (I) and (II) at another point in time
IV) Analysis of the images obtained.

In another preferred embodiment of this invention, the metal complexes are used for in vitro detection of amyloid plaques in cerebral tissue. On the one hand, the metal complexes that can be used according to the invention can be used directly therein by being incubated with cerebral tissue, such as, for example, fixed sections, and the binding of the metal complexes to the plaques and thus the localization of the plaques are detected by a suitable imaging method. The imaging method that is used depends on the bonded metal ion. If the metal ion is paramagnetic, the detection or the localization can be done by an NMR-based method; in the case of radioactive metal ions (or radioactive elements in the skeleton), the radiodiagnosis can be used. The metal complexes that can be used according to the invention can also be detected with the aid of an antibody that specifically detects these metal complexes. In this case, the antibody carries either one group itself, which makes direct detection possible, or a second antibody is used, which detects the first antibody, and which for its part makes possible the detection, for example, via a fluorescence labeling or an enzyme-mediated color reaction. Such methods are known in general to one skilled in the art.

It is also possible, however, as described in Example 2, that the metal complexes that can be used according to the invention contain a signaling group, for example a covalently bonded fluorescence dye. In this case, the detection is carried out after incubation also by a correspondingly adapted imaging method. In Example 2, in which the fluorescence dye carbocyanine is contained in a metal complex that can be used according to the invention, the detection is carried out by detecting fluorescence.

This invention thus also comprises a process for in-vitro detection of amyloid plaques and/or in-vitro diagnosis of Alzheimer's disease, which comprises at least the following steps:
(a) Incubation of a metal complex, usable according to the invention, that contains a signaling group, with optionally pretreated and/or fixed cerebral tissue of patients, as well as
(b) Detection of amyloid plaques and/or localization and/or quantification of amyloid plaques with a suitable detection method or imaging method.

In particular, the detection method or imaging method is selected from SPECT, PET, ¹⁸F-MRI and optical imaging.

This invention thus also comprises a process for in-vitro detection of amyloid plaques and/or the in-vitro diagnosis of Alzheimer's disease, which comprises at least the following steps:
(a) Incubation of a metal complex, usable according to the invention, with optionally pretreated and/or fixed cerebral tissue of patients; and
(b) Detection of amyloid plaques and/or localization and/or quantification of amyloid plaques with a suitable detection method, selected in particular from MR spectroscopy, MRI and x-ray-based methods.

In addition, the invention relates to a kit for diagnosis of Alzheimer's disease that contains at least one metal complex, usable according to the invention, with a signaling group.

In addition, the invention relates to a kit for diagnosis of Alzheimer's disease that contains at least one metal complex that can be used according to the invention.

“Chelating agent” in terms of this invention is a complexing substance that forms a complex with a stability constant of at least 10¹⁵, preferably at least 10¹⁸, with at least one metal ion of atomic numbers 21-29, 31-33, 37-39, 42-44, 49 or 57-83. The stability constant is determined as described in Martell, A. E. ; Motekaitis, R. J. (The Determination and Use of Stability Constants, 2nd Ed.; VCH: New York, 1992).

TABLE 1
Metal Complexes (MK) That are Quite Especially Preferably Used
According to the Invention, Their Origin and Their Physicochemical
Parameters

| | | Example | | | |
|---|---|---|---|---|---|
| Complex | Origin | No. | R₁ [l/mmol * s] | CMC [mol/l] | 2 Rh [nm] |
| MK 1 | WO 99/01161 | 18 | 23.0 | 1.5 10⁻⁴ | 3.5 |
| MK 2 | WO 97/26017 | 1 | 29.7 | 1.0 10⁻⁵ | 31.5 |
| MK 3 | WO 97/26017 | 2 | 33.0 | 2.3 10⁻⁵ | 14.0 |
| MK 4 | WO 97/26017 | 3 | 27.5 | 1.44 10⁻⁵ | 3.2 |
| MK 5 | WO 99/01161 | 25 | 15.1 | 3.1 10⁻⁵ | 7.0 |
| MK 6 | WO 97/26017 | 31 | 26.0 | 9.8 10⁻⁴ | 4.3 |
| MK 7 | WO 99/01161 | 12 | 21.4 | 1.81 10⁻⁶ | 4.2 |
| MK 8 | WO 97/26017 | 33 | 35.7 | 1.86 · 10⁻⁶ | 4.6 |
| MK 9 | WO 97/26017 | 35 | 34.0 | 3.25 · 10⁻⁶ | 4.3 |
| MK 10 | WO 97/26017 | 34 | 24.9 | 7.06 · 10⁻⁶ | 3.2 |
| MK 11 | WO 97/26017 | 32 | 24.8 | 2.88 · 10⁻⁶ | 35.5 |
| MK 12 | WO 99/01161 | 1 | 19.5 | 8.9 · 10⁻⁴ | 2.2 |
| MK 13 | WO 02/13874 | 21 | 15.9 | 2.5 · 10⁻⁶ | 4.4 |
| MK 14 | WO 02/13874 | 54 | 21.3 | 3.9 10⁻⁵ | 4.9 |
| MK 15 | WO 99/01161 | 14 | 19.3 | 8.7 · 10⁻⁶ | 3.2 |
| MK 16 | WO 00/56723 | 7 | 21.0 | 2.8 · 10⁻⁶ | 4.3 |
| MK 17 | WO 02/13874 | 6 | 13.3 | 2.65 · 10⁻⁶ | 6.0 |
| MK 18 | WO 02/13874 | 2 | 19.6 | 3.9 · 10⁻⁶ | 4.4 |
| MK 19 | WO 02/13874 | 5 | 30.3 | 5.2 · 10⁻⁵ | 3.0 |
| MK 20 | WO 00/56723 | 4 | 21.9 | 4.6 10⁻⁵ | 5.5 |
| MK 21 | WO 02/13874 | 3 | 21.2 | 2.92 10⁻⁵ | 2.5 |
| MK 22 | WO 00/56723 | 7 | 27.8 | 4.4 · 10⁻⁶ | 5.7 |
| MK 23 | WO 02/13874 | 1 | 25.7 | 7.9 · 10⁻⁶ | 5.4 |
| MK 24 | WO 99/01161 | 1 | 13.9 | 6.3 · 10⁻⁶ | 10.0 |
| MK 25 | WO 99/01161 | 5 | 21.3 | 1.4 · 10⁻⁴ | 3.5 |
| MK 26 | WO 02/13874 | 57 | 22.8 | 4.3 · 10⁻⁶ | 5.2 |
| MK 27 | WO 97/25017 | 38 | 30.5 | 1.07 · 10⁻⁵ | 7.4 |
| MK 28 | DE102005033902.6- | 14 | 25.5 | 1.73 10⁻⁴ | 8.4 |
| | 44 | | | | |

CMC: Critical Micelle Formation Concentration
2 Rh: Hydrodynamic Micelle Diameter
R₁: Relaxivity
The measurements were made in plasma at 40° C. and a magnetic field strength of 0.47 Tesla.

### BRIEF DESCRIPTION OF DRAWINGS

FIG. 1 and 2 shown MRI images of mice brains in Alzheimer's models.

### EXAMPLES

### Example 1

### MRT Visualization of Alzheimer Lesions After Intravenous Administration of Contrast Medium in APP-23 Mice

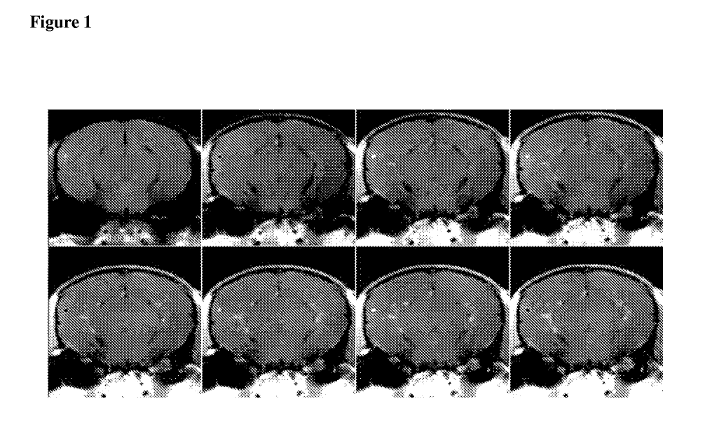

The images of FIG. 1 show MR images of the brain before as well as up to 60 minutes after intravenous administration of 0.5 mmol of Gd/kg of body weight of metal complex 13 (MK13) in APP-23 mice (mice with genetically-induced Alzheimer's disease). The T1-weighted spin echo images (Bruker, 7T, 2D-T1w-SE; TR/TE=600/11 ms; NA=4; TA=8 min) illustrate a strong signal increase in the amyloid plaques of the diseased brains. The localization of the amyloid-containing structures was confirmed by means of Puchtler's Congo red stain. With this test, the suitability of the compounds according to the invention as in-vivo markers for amyloid plaques in the brains of mice with Alzheimer's disease could be shown.

### Example 2

### Fluorescence-Visualization of Alzheimer Lesions in the Histopathological Preparation of the Brain of an APP-23 Mouse, 26 Hours After Intravenous Administration of the Fluorescence-Labeled Contrast Medium; Correlation with Histological Detection of Amyloid Plaques

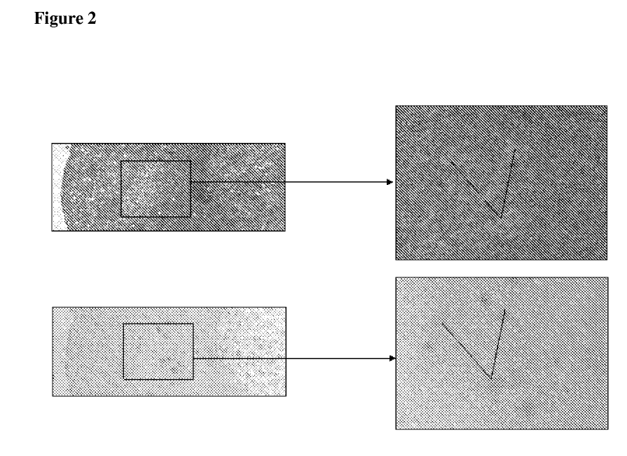

The figures show microscopic fluorescence images of the brain of an APP-23 mouse 26 hours after intravenous administration of 0.5 mmol of Gd/kg of body weight of the carbocyanine-labeled metal complex 13 (MK13) (FIG. 2, upper series), as well as the corresponding coloring of amyloid plaques by means of Puchtler's Congo red stain (FIG. 2, lower series). The images illustrate a strong fluorescence signal of the contrast medium in the amyloid plaques of the diseased brains. The localization of the compound according to the invention in the amyloid-containing structures was confirmed by means of Puchtler's Congo red stain. With this test, the suitability of the compounds according to the invention as in-vivo and/or in-vitro markers for amyloid plaques in the brains of mice with Alzheimer's disease could be shown.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2006 021 495.1, filed May 9, 2006, and U.S. Provisional Application Ser. No. 60/798,709 filed May 9, 2006 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of diagnosing for the production of amyloid-containing plaques in a sample comprising contacting said sample with a metal chelate containing
at least one perfluorinated alkyl radical, and
at least one chelating agent radical, and
at least one metal ion equivalent of atomic numbers 21-29, 31-33, 37-39, 42-44, 49 or 57-83, as well as salts thereof.

2. A method according to claim 1, characterized in that the metal chelates or salts thereof contain
1 perfluorinated alkyl radical, and
1 or 2 chelating agent radicals.

3. A method according to claim 1, wherein the perfluorinated alkyl radical contains 4-30 C atoms, preferably 6-20 C atoms.

4. A method according to claim 1, wherein the metal ion is paramagnetic.

5. A method according to claim 4, wherein the metal complexes contain at least a divalent or trivalent ion of the elements of atomic numbers 21-29, 42, 44 and 58-70.

6. A method according to claim 4, wherein the metal complexes or salts thereof have
a critical micelle formation concentration<10⁻³ mol/l,
a hydrodynamic micelle diameter (2 Rh)>1 nm, and
a proton relaxivity in plasma (R₁)>10 l/mmol*s.

7. A method according to claim 4, wherein the metal complexes are used as MRI contrast media for diagnosis of Alzheimer's disease in a patient.

8. A method according to claim 1, wherein the metal complexes are used as contrast media for differentiation between Alzheimer's disease and other dementia diseases in a patient.

9. A method according to claim 1, wherein the metal complexes are used as contrast media for early detection of Alzheimer's disease in a patient.

10. A method according to claim 1, wherein the metal complexes are used as contrast media for monitoring the course of treatment in treated Alzheimer patients.

11. A method according to claim 1, wherein the metal complexes are used as contrast media for identifying patients at risk for developing Alzheimer's disease.

12. A method according to claim 6, wherein metal complexes are used whose micelle formation concentration is <10⁻⁴ mol/l.

13. A method according to claim 6, wherein metal complexes are used whose hydrodynamic micelle diameter is ≧3 nm, preferably >4 nm.

14. A method according to claim 4, wherein metal complexes are used that have a proton relaxivity in the plasma of >13 l/mmol*s, preferably >15 l/mmol*s.

15. A method according to claim 1, wherein the metal complexes overcome the blood-brain barrier.

16. A method according to claim 1, wherein the metal complexes contain at least one divalent or trivalent ion of the elements of atomic numbers 25, 26, 39 or 57-83.

17. A method according to claim 16 for use in x-ray diagnosis.

18. A method of visualizing amyloid-containing plaques in a sample in particular for use in SPECT, PET, ¹⁸F-MRI or optical imaging comprising contacting said sample with a metal chelate containing
at least one perfluorinated alkyl radical, and
at least one chelating agent radical, and
at least one metal ion equivalent of the atomic numbers 21-29, 31-33, 37-39, 42-44, 49 or 57-83, as well as salts thereof,
and a signaling group.

19. Process for the in-vitro detection of amyloid plaques and/or the in-vitro diagnosis of Alzheimer's disease containing the following steps:
(a) Incubation of a metal complex that can be used according to the invention, with optionally preferred and/or fixed cerebral tissue of patients;
(b) Detection of amyloid plaques and/or localization and/or quantification of amyloid plaques with a suitable detection method, in particular selected from MR spectroscopy, MRI, and x-ray-based methods.

20. Process for the in-vitro detection of amyloid plaques and/or the in-vitro diagnosis of Alzheimer's disease containing the following steps:
(a) Incubation of a metal complex that can be used according to the invention, containing a signaling group, with optionally pretreated and/or fixed cerebral tissue of patients, as well as
(b) Detection of amyloid plaques and/or localization and/or quantification of amyloid plaques with a suitable detection method.
